(19) United States
(12) Patent Application Publication
(10) Pub. No.: US 2024/0024431 A1
(43) Pub. Date: Jan. 25, 2024

(54) TISSUE-DERIVED MATRIKINE COMPOSITIONS AND METHODS THEREFOR

(71) Applicant: XYLYX BIO, INC., Brooklyn, NY (US)

(72) Inventors: John D. O'NEILL, Brookklyn, NY (US); Igal GERMANGUZ, Brooklyn, NY (US); Evelyn ARANDA, Brooklyn, NY (US); Jennifer XIONG, Brooklyn, NY (US); Natalia KISSEL, Brooklyn, NY (US); Alexandra NICHOLS, Brooklyn, NY (US); Eddie GADEE, Brooklyn, NY (US); Richard NGUYEN, Brooklyn, NY (US); Drew DALY, Brooklyn, NY (US); Andrea NYE, Brooklyn, NY (US)

(21) Appl. No.: 18/021,660

(22) PCT Filed: Sep. 8, 2021
(86) PCT No.: PCT/US2021/049432

§ 371 Date: Feb. 16, 2023

Related U.S. Application Data
(60) Provisional application No. 63/075,638, filed on Sep. 8, 2020.

(51) Int. Cl. A61K 38/39; A61K 9/00; A61K 31/727; A61K 8/65; A61K 8/64; A61K 8/73; A61Q 19/08; A61P 17/10; A61P 17/02

(52) U.S. Cl. CPC A61K 38/39; A61K 9/0014; A61K 31/727; A61K 8/65; A61K 8/64; A61K 8/735; A61Q 19/08; A61P 17/10; A61P 17/02

(57) ABSTRACT

A composition for topical administration to an epithelium is disclosed. The composition comprises a deconstructed matrisome including one or more enzymatically fragmented peptides derived from biological tissue. The composition further comprises one or more pharmaceutically acceptable or cosmetically acceptable excipients. The deconstructed matrisome may be fragmented through degradation by one or more enzymes to produce the one or more peptides. The one or more peptides may be configured to retain cell signaling ability, thereby promoting one or more of tissue homeostasis, tissue repair, and tissue regeneration. The composition may be used to treat a tissue such as skin tissue exhibiting scarring, acne, eczema, psoriasis, and other skin conditions.

### CROSS-REFERENCE TO RELATED APPLICATIONS

This application claims the benefit of priority to U.S. Provisional Application No. 63/075,638 entitled “Tissue-Derived Matrikine Compositions and Methods Therefor,” filed Sep. 8, 2020, which is incorporated herein by reference in its entirety.

### TECHNICAL FIELD

This present disclosure relates generally to compositions comprising matrisome components and related methods. The disclosed compositions and methods may be utilized, for example, to treat scars and other skin conditions, such as acne, eczema, and psoriasis.

### BACKGROUND

Dermal tissue damage affects 500+ million people each year worldwide, while minimal options for effective topical treatment are commercially available. Current top-selling over-the-counter scar treatment products are limited to plant-based or chemical ingredients with no active repair or regenerative signaling capacity. There is a lack of products with bioactive ingredients that offer proven support for skin repair—a significant market gap waiting to be filled.

Existing products to treat scar repair incorporate herbal or food extracts or silicone-based or other synthetic ingredients but show limited data around proven regenerative bioactivity or the ability to tailor to specific skin condition, ethnicities, ages, etc. to facilitate “personalized” product offerings. There is a significant market opportunity for a topical skin bioactive that can prevent and effectively reduce redness and scars after injury by replicating natural regenerative signals.

In its native environment, extracellular matrix (ECM) is a scaffold with tissue-specific cues (e.g., molecular, structural, biomechanical) that provides structure for cell maintenance and growth and mediates cell proliferation, differentiation, gene expression, migration, orientation, and assembly. ECM comprises an interlocking mesh of components including but not limited to viscous proteoglycans (e.g., heparin sulfate, keratin sulfate, and chondroitin sulfate) that provide cushioning, collagen and elastin fibers that provide strength and resilience, and soluble multiadhesive proteins (e.g., fibronectin and laminin) that bind the proteoglycans and collagen fibers to cell receptors. Native extracellular matrix also commonly includes hyaluronic acid and cellular adhesion molecules (CAMs) such as integrins, cadherins, selectins, and immunoglobulins.

The complexity of the ECM has proven difficult to recapitulate in its entirety outside of its native environment. Mimicking ECM structure using synthetic biomaterials or mimicking composition by adding purified ECM components is possible. While offering structural mimics, synthetic biomaterials can alter cell behavior (i.e., proliferation, differentiation, gene expression, migration, orientation, and assembly) in vitro and potentially generate cytotoxic by-products at the site of implantation, leading to poor wound healing or an inflammatory environment.

Further, the ECM of each type of tissue may comprise a different mixture of components, concentrations of components, and/or properties that are suited to the tissue's unique set of roles and may signal unique cell activity. Further, disease states in tissues may be associated with specific alterations in the biochemical composition, structure, and biomechanics of the ECM environments.

As such, it would be advantageous to have a topical composition capable of activating regenerative bioactivity in tissue-specific and/or population-specific manners by recapitulating in vivo niche environments.

### SUMMARY

This summary is provided to comply with 37 C.F.R. § 1.73. It is submitted with the understanding that it will not be used to interpret or limit the scope or meaning of the present disclosure.

Embodiments of the invention are directed to a composition for topical administration to an epithelium, the composition comprising: a deconstructed matrisome including one or more enzymatically fragmented peptides derived from a at least one biological tissue; and one or more pharmaceutically acceptable or cosmetically acceptable excipients, wherein the deconstructed matrisome is in an amount from about 0.1% by weight to about 15% by weight of the composition; and wherein the one or more enzymatically fragmented peptides are configured to retain cell signaling ability, thereby promoting one or more of tissue homeostasis, tissue repair, and tissue regeneration.

Embodiments of the invention are directed to a method of promoting repair or regeneration in a target tissue, the method comprising: topically administering a composition to an epithelium of the target tissue, the composition comprising: a deconstructed matrisome including one or more enzymatically fragmented peptides derived from at least one biological tissue, and one or more pharmaceutically acceptable or cosmetically acceptable excipients, wherein the deconstructed matrisome is in an amount from about 0.1% by weight to about 15% by weight of the composition; and wherein the one or more enzymatically fragmented peptides are configured to retain cell signaling ability, thereby promoting one or more of tissue homeostasis, tissue repair, and tissue regeneration in the target tissue.

Embodiments of the invention are directed to a method of increasing keratin gene expression in a target tissue, the method comprising: topically administering a composition to an epithelium of the target tissue, the composition comprising: a deconstructed matrisome including one or more enzymatically fragmented peptides derived from at least one biological tissue, and one or more pharmaceutically acceptable or cosmetically acceptable excipients, wherein the deconstructed matrisome is in an amount of about 0.1% by weight to about 15% by weight of the composition; and wherein the one or more enzymatically fragmented peptides are configured to retain cell signaling ability, thereby reducing or preventing one or more of laxity, wrinkling, and sagging in the target tissue.

Embodiments of the invention are directed to a method of reducing dermal redness in a target tissue, the method comprising: topically administering a composition to an epithelium of the target tissue, the composition comprising: a deconstructed matrisome including one or more enzymatically fragmented peptides derived from at least one biological tissue, and one or more pharmaceutically acceptable or cosmetically acceptable excipients, wherein the deconstructed matrisome is in an amount of about 0.1% by weight to about 15% by weight of the composition; and wherein the one or more enzymatically fragmented peptides are configured to retain cell signaling ability, thereby promoting healing or recovery in the target tissue from one or more of scar formation, a wound, and a burn.

Embodiments of the invention are directed to a method of lowering the pH of a surface of a target tissue, the method comprising: topically administering a composition to an epithelium of the target tissue, the composition comprising: a deconstructed matrisome including one or more enzymatically fragmented peptides derived from at least one biological tissue, and one or more pharmaceutically acceptable or cosmetically acceptable excipients, wherein the deconstructed matrisome is in an amount of about 0.1% by weight to about 15% by weight of the composition; and wherein the one or more enzymatically fragmented peptides are configured to retain cell signaling ability, thereby lowering the pH of the surface and reducing presence or growth of a pathogen on the surface of the target tissue.

Embodiments of the invention are directed to a method of improving a characteristic of a target skin tissue, the method comprising: topically administering a composition to an epithelium of the target skin tissue, the composition comprising: a deconstructed matrisome including one or more enzymatically fragmented peptides derived from at least one biological tissue, and one or more pharmaceutically acceptable or cosmetically acceptable excipients, wherein the deconstructed matrisome is in an amount of about 0.1% by weight to about 15% by weight of the composition; wherein the one or more enzymatically fragmented peptides are configured to retain cell signaling ability, thereby improving the characteristic of the target skin tissue; and wherein the characteristic of the target skin tissue is selected from the group consisting of firmness, elasticity, fine lines, wrinkles, skin texture, skin tone, and appearance.

Embodiments of the invention are directed to a method of increasing cell regeneration in a target tissue, the method comprising: topically administering a composition to an epithelium of the tissue, the composition comprising: a deconstructed matrisome including one or more enzymatically fragmented peptides derived from at least one biological tissue, and one or more pharmaceutically acceptable or cosmetically acceptable excipients, wherein the deconstructed matrisome is in an amount of about 0.1% by weight to about 15% by weight of the composition; and wherein the one or more enzymatically fragmented peptides are configured to retain cell signaling ability, thereby restoring an epithelial barrier of the target tissue.

### BRIEF DESCRIPTION OF THE DRAWINGS

The accompanying drawings, which are incorporated in and form a part of the specification, illustrate the embodiments of the invention and together with the written description, serve to explain the principles, characteristics, and features of the invention. In the drawings:

FIG. 1 depicts an illustrative diagram of a method of making a fibrosis-specific extracellular matrix substrate in accordance with an embodiment in accordance with an embodiment.

FIG. 2 depicts an example of normal human dermal fibroblasts cultured on deconstructed matrisome in accordance with an embodiment.

FIG. 3 depicts an example of normal human dermal fibroblasts cultured on deconstructed matrisome in accordance with an embodiment.

FIG. 4 depicts an example of an SDS-PAGE gel of blood vessel and skin extracellular matrix components in accordance with an embodiment.

FIG. 5 depicts an example of gene expression in extracellular matrix components cultured with tissue-specific cells in accordance with an embodiment.

FIG. 6 depicts an example of biochemical analysis of blood vessel and skin extracellular matrix components in accordance with an embodiment.

FIG. 7 depicts an example of a matrikine-induced increase in dermal tissue regeneration in accordance with an embodiment.

FIG. 8 depicts an example of a matrikine-induced reduction in redness during wound healing in accordance with an embodiment.

FIG. 9 depicts an example of matrikine-induced reduction in wound size in accordance with an embodiment.

FIG. 10 depicts an illustration of matrikine-induced healing in accordance with an embodiment.

FIG. 11 depicts an example of matrikine-induced cellular migration and wound surface reduction in accordance with an embodiment.

FIGS. 12A and 12B depicts an example of a human repeat insult patch test in accordance with an embodiment.

FIG. 13 depicts an example of matrikine-induced wound healing in accordance with an embodiment.

FIG. 14 depicts an example of matrikine-induced wound healing in accordance with an embodiment.

FIG. 15 depicts an example of matrikine-induced wound healing in accordance with an embodiment.

FIG. 16 depicts an example of matrikine-induced scar reduction in accordance with an embodiment.

FIG. 17 depicts an example of matrikine-induced scar reduction in accordance with an embodiment.

FIG. 18 depicts an example of matrikine-induced scar reduction in accordance with an embodiment.

FIG. 19 depicts an example of matrikine-induced scar reduction in accordance with an embodiment.

FIG. 20 depicts an example of matrikine-induced treatment of acne vulgaris in accordance with an embodiment.

FIG. 21 depicts an example of matrikine-induced treatment of acne vulgaris in accordance with an embodiment.

FIG. 22 depicts an example of matrikine-induced treatment of wrinkles in accordance with an embodiment.

FIG. 23 depicts an illustration of user perception in accordance with an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope. Such aspects of the disclosure be embodied in many different forms; rather, these embodiments are provided so that this disclosure will be thorough and complete, and will fully convey its scope to those skilled in the art.

Various aspects will be described in detail hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of.”

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

All percentages, parts and ratios are based upon the total weight of the formulations and compositions and all measurements made are at about 25° C., unless otherwise specified.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

The terms “patient” and “subject” are interchangeable and may be taken to mean any living organism which may be administered and/or treated with compounds or compositions provided for herein. As such, the terms “patient” and “subject” may comprise, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child, or infant. In some embodiments, the patient or subject is a human.

Generally speaking, the term “tissue” refers to any aggregation of similarly specialized cells which are united in the performance of a particular function. As used herein, “tissue”, unless otherwise indicated, refers to tissue which includes elastin as part of its necessary structure and/or function. For example, connective tissue which is made up of, among other things, collagen fibrils and elastin fibrils satisfies the definition of “tissue” as used herein. Additionally, elastin appears to be involved in the proper function of blood vessels, veins, and arteries in their inherent visco-elasticity.

The term “composition” as used herein refers to a combination or a mixture of two or more different ingredients, components, or substances.

The term “matrikine” as used herein refers to extracellular matrix-derived peptides which regulate cell activity. Matrikines are bioactive peptides generated as a result of partial enzymatic degradation (i.e., proteolysis) of extracellular matrix macromolecules. The peptides are fragments of the full-length molecules and often modulate cellular activities through signaling in different manners than the full-length molecules, including but not limited to modulating cell proliferation, migration, and apoptosis. Furthermore, the signaling from matrikines may vary based the degree of fragmentation that occurs through enzymatic degradation (i.e. the size of the fragments). The term “Matrikyne” as used here refers to the formulations of matrikines disclosed in the present application and/or produced by XYLYX BIO, INC. of New York, USA.

The term “matrisome” as used herein refers to the set of matrikines released from an extracellular matrix or resulting from the enzymatic degradation of an extracellular matrix. A complete matrisome may comprise different concentrations of matrikines depending on the tissue-specific extracellular matrix the matrisome is derived from and represents a full complement of matrikines.

The term “excipients” as used herein encompasses carriers and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “keratinous fiber” as used herein refers to any tissue which contain keratin as a fibrous structural protein, including, but not limited to, skin, hair, and nails.

The terms “topically” and “topical” as used herein refer to application of the compositions to the surface of the skin, mucosal cells, keratins and tissues. Examples of keratins are nails and hair.

The phrase “pharmaceutically acceptable” or “cosmetically acceptable” is employed herein to refer to those agents of interest/compounds, salts, compositions, dosage forms, etc., which are within the scope of sound medical judgment suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g., animals), and more particularly, in humans.

The term “cosmetic” means an agent utilized, and/or intended to be applied to the human body for cleansing, beautifying, promoting attractiveness, altering the appearance of the skin or any combination thereof.

As used herein, the term “therapeutic” means an agent utilized to treat, combat, ameliorate, prevent, or improve an unwanted condition or disease of a patient. In part, embodiments of the present invention are directed to the treatment of wounds, scars, and skin conditions.

In some embodiments, the compounds and methods disclosed herein can be utilized with or on a subject in need of such treatment, which can also be referred to as “in need thereof” As used herein, the phrase “in need thereof” means that the subject has been identified as having a need for the particular method or treatment and that the treatment has been given to the subject for that particular purpose.

The term “treat,” “treated,” or “treating” as used herein, refers to methods of treating a skin disorder or a systemic condition, and generally includes the administration of a compound or composition, wherein the object is to reduce the frequency of, or delay the onset of, symptoms of a medical condition, enhance the texture, appearance, color, sensation, or hydration of the intended tissue treatment area of the tissue surface in a subject relative to a subject not receiving the compound or composition, or to otherwise obtain beneficial or desired clinical results. For the purposes of this invention, beneficial or desired clinical results include, but are not limited to, reversal, reduction, or alleviation of symptoms of a condition; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “inhibiting” includes the administration of a compound of the present invention to prevent the onset of the symptoms, alleviating the symptoms, reducing the symptoms, delaying or decreasing the progression of the disease and/or its symptoms, or eliminating the disease, condition or disorder.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As discussed herein, pro-regeneration biomaterial peptides derived from extracellular matrix (i.e., matrikines) can provide cells with the complex structural support and biochemical signals required for cellular regeneration. Bioactive compositions may provide comprehensive skin repair and overall skin health using regenerative bioactive ingredients. The compositions may be varied to create a product line that includes products aimed at scar treatment and other skin conditions, such as acne, eczema, and psoriasis (e.g., Matrikyne® compositions available from XYLYX BIO, INC.).

Hundreds of matrikine bioactive protein complexes promote skin cell regeneration and healing, thereby providing a clinically proven ingredient with proven rejuvenating properties. The bioactive components orchestrate essential communication between skin cells, the healthy skin microbiome and the immune system, integrally supporting active skin cell regeneration and health. The hundreds of matrikine bioactive protein complexes come from six broad families of bioactive molecules that work together to contribute essential repair functions. Bioactive families and their benefits include: laminins (regulate cell migration and differentiation), proteoglycans (signal skin cell renewal and proliferation), growth factors (support skin cell rejuvenation), glycoproteins (direct immune cells and system), elastin (support in tissue integrity and tone), collagens (support skin repair and regeneration).

Natural matrikine bioactivity supports tissue repair and healing by regulating inflammation, inhibiting fibrosis and enhancing production of collagen and expression of growth factors. Matrikines guide cellular regeneration and are integral to physiological ‘dialogue’ underlying skin health. Unlike conventionally available products, fragmented extracellular matrix includes matrikines and is able to penetrate the skin to facilitate dialogue between skin cells, the immune system and the microbiome. Formulations can be targeted to various applications, including reduction in the appearance of fine lines and wrinkles, scar and acne treatment, and overall skin brightening, firming and rejuvenation. Matrikines are also proven to reduce redness by restoring the skin's natural balance and can be used for scar treatment, redness reduction, eczema, roseacea and as an anti-wrinkle treatment. Furthermore, matrikines have the ability to tailor to skin condition, age ethnicity, etc. for product personalization.

### Topical Matrikine Compositions

Embodiments disclosed herein are directed to a composition for topical application comprising fragmented deconstructed matrisome including one or more peptides (i.e., matrikines). The composition may further comprise one or more excipients (e.g., a pharmaceutically acceptable excipient or a cosmetically acceptable excipient). In some embodiments, the deconstructed matrisome may be fragmented to generate the one or more peptides through proteolysis (i.e., enzymatic degradation) of one or more deconstructed matrisome components. Further, the one or more peptides may be configured to promote one or more of tissue homeostasis, tissue repair, and tissue regeneration.

The matrikines may be may be derived from tissue-specific extracellular matrix (TS-ECM) specific to a variety of tissue types, and thus the resulting mixture of matrikines in the composition may emulate the niche environment of various tissues. In some embodiments, the TS-ECM may comprise skin-specific ECM. However, a variety of TS-ECMs may be used to derive the matrikines. In some embodiments, the TS-ECM may be selected from blood vessel-specific ECM, cartilage-specific ECM, intestine-specific ECM, liver-specific ECM, placenta-specific ECM, skin-specific ECM and stomach-specific ECM. In still additional embodiments, the TS-ECM may emulate a niche environment specific to another tissue. For example, the tissue may be selected from the adrenal gland, amnion, bladder, bone, brain, breast, chorion, connective tissue, esophagus, eye, fat, heart, kidney, larynx, ligament, lung, lymph, microvasculature, muscle, mouth, omentum, ovary, fallopian tube, thyroid, parathyroid, large intestine, small intestine, pancreas, peritoneum, pharynx, placenta membrane, prostate, rectum, smooth muscle, spinal cord, spinal fluid, spleen, tendon, testes, thymus, umbilical cord, uterus, vagina, or Wharton's Jelly. In some embodiments, the TS-ECM may emulate a region of the anatomy, an organ, or a region of an organ. For example, a TS-ECM may represent the large intestine or it may more specifically represent the colon or the rectum.

In some embodiments, the deconstructed matrisome may be derived from biological tissue. A variety of tissue sources may be used as a starting material to obtain deconstructed matrisome. In some embodiments, the tissue source is a human source. In some embodiments, the tissue source is an animal source. For example, the tissue may be porcine (i.e., sourced from a pig) or any other animal tissue known to have clinical relevance.

In some embodiments, the tissue source may be other than a human or animal source. In some embodiments, the tissue source may be selected from a plant source and a fungal source. While the deconstructed matrisomes exemplified herein are representative of animal tissues, the selected tissue may vary based on the type of organism selected as a tissue source. For example, where the tissue source is a plant source and/or a fungal source, the selected tissue may be any tissue types known to be present in plants and/or fungi as understood by a person having an ordinary level of skill in the art.

In some embodiments, the tissue source is selected from fetal tissue, juvenile tissue, and adult tissue. In some embodiments, the tissue source is healthy tissue. However, in some embodiments, the tissue source is selected from diseased tissue, transgenic tissue, or tissue having a specific disorder or health condition. For example, specific disorders or conditions may result in overexpression of specific proteins and/or peptides. Accordingly, it may be beneficial to apply a composition comprising the specific proteins or matrikines to an individual exhibiting under expression of the same specific proteins or matrikines. Therefore, it may be advantageous to derive the matrikines from TS-ECM from a non-healthy tissue in a targeted manner. The resulting TS-ECM is representative of extracellular matrix from the tissue source, or more generally from tissue having the same relevant characteristics as the tissue source (e.g., fetal human skin tissue will yield skin-specific ECM representative of fetal human skin).

In some embodiments, the matrikines are isolated from a single TS-ECM. In some embodiments, the matrikines are isolated from a plurality of TS-ECMs. Accordingly, the mixture of matrikines and relative concentrations of matrikines included in the composition may represent a single tissue a combination of tissues. The plurality of TS-ECMs may be provided in predetermined quantities or ratios with respect to one another in order to tailor the resulting mixture of matrikines. Further, any number of matrikines isolated from the TS-ECM may be adjusted (e.g., adjusting a concentration) or entirely removed in order to produce a composition having a matrikine mixture with desired properties for tissue repair and regeneration.

In some embodiments, the composition includes enzymatically fragmented deconstructed matrisome and/or the matrikines in specified concentrations that emulates the extracellular environment found in a specific native tissue. The composition may include a specific combination of deconstructed matrisome macromolecules such as scaffolding proteins, ECM-associated proteins, ECM regulators, glycoproteins, proteoglycans, laminins, extracellular matrix associate proteins, soluble growth factors, inflammatory cytokines and chemokines, immune mediators, and secreted factors in the extracellular fluid. Additionally, the composition may include a specific combination of matrikines. Because matrikines are derived from the macromolecules in the TS-ECM (i.e., ‘parent’ proteins or macromolecules), the mixture of matrikines (i.e., ‘child’ peptides) in the composition may vary according to the makeup of the TS-ECM. Therefore, the matrikines in the composition may correspond to the type of TS-ECM from which the matrikines are sourced. Further, each of these components may have subtypes, the presence of each of which may vary from one deconstructed matrisome to another deconstructed matrisome. Each deconstructed matrisome may be characterized by the presence or absence of one or more components. Further, the concentration of each component may vary from one deconstructed matrisome to another deconstructed matrisome.

As described above, ECM comprises macromolecules (e.g., proteins. lipids, and polysaccharides) and other factors that are specific for cell-signaling in a particular niche-environment. In native ECM, the ECM components form a three-dimensional ultrastructure. In some applications, one may prefer a more uniform substrate like a solution or a hydrogel, for example, for topical application. The TS-ECM produced by the methods described herein is distinct from native ECM. The TS-ECM is decellularized and the removal of the cellular structure modulates the concentrations of macromolecules and other cell-signaling factors. Further, the three-dimensional ultrastructure may be removed and the various components of the deconstructed matrisome may be digested into fragments. Any of the ECM components in the various embodiments and/or formulas described herein may be fragmented in the deconstructed matrisome, including but not limited to collagen, elastin, glycosaminoglycans, proteoglycans, matrix associated factors, ECM regulators, matrisome secreted factors, immune factors, marrow associated factors, and other structural factors. The removal of the three-dimensional ultrastructure of the ECM and the fragmentation of deconstructed matrisome components facilitates formation of a homogenous mixture for use in forming compositions for topical application, e.g., hydrogels, liquid solution, powders, and other formats as described herein. Surprisingly, the fragmented components nonetheless contribute to cell signaling along with small molecules, thus retaining the characteristics of the niche environment to a high degree despite the fragmentation and lack of ultrastructure which are needed to form the conventional substrate structure.

In some embodiments, the deconstructed matrisome comprises a homogenous mixture of macromolecule fragments including collagen, elastin, and glycosaminoglycan. In some embodiments, the deconstructed matrisome comprises macromolecules or macromolecule fragments including collagen, elastin, and glycosaminoglycan, wherein the amount of each macromolecule may be decreased after decellularization. In some embodiments, the deconstructed matrisome comprises a homogenous mixture of macromolecule fragments including collagen, elastin, and glycosaminoglycan, wherein the concentration of each macromolecule may be changed after decellularization.

In some embodiments, the deconstructed matrisome comprises a homogenous mixture of macromolecule fragments. In some embodiments, the deconstructed matrisome comprises a homogenous mixture of macromolecule fragments, wherein the macromolecules may be fully or partially fragmented after enzymatic digestion. In some embodiments, the deconstructed matrisome comprises a homogenous mixture of macromolecule fragments, wherein the homogenous mixture retains cellular signaling. In some embodiments, the deconstructed matrisome comprises a homogenous mixture of macromolecule fragments, wherein the homogenous mixture does not contain the ECM three-dimensional ultra-structure. In some embodiments, the ECM three-dimensional ultra-structure is not required for cell-matrix recognition. In some embodiments, interactions responsible for cell-matrix recognition is not limited to structural cues from acellular matrix, but also relies on signaling from small molecules or protein fragments. In some embodiments described herein, the deconstructed matrisome is processed into an ECM powder. In some embodiments, the ECM powder comprises a homogenous mixture of macromolecule fragments, wherein the macromolecules may be fully or partially fragmented after enzymatic digestion. In some embodiments, the ECM powder comprises a homogenous mixture of macromolecule fragments, wherein the homogenous mixture retains cellular signaling. In some embodiments, the ECM powder comprises a homogenous mixture of macromolecule fragments, wherein the homogenous mixture does not contain the ECM three-dimensional ultra-structure. In some embodiments, the ECM three-dimensional ultra-structure is not required for cell-matrix recognition. In some embodiments, interactions responsible for cell-matrix recognition is not limited to structural cue from decellularized matrix, but also relies on signaling from small molecules or protein fragments.

In some embodiments, the TS-ECM may not be enzymatically digested and the three-dimensional ultrastructure may be maintained, e.g., as an acellular and/or dehydrated scaffold.

The composition may be characterized by any matrikine components, concentrations thereof, and/or changes thereof from normal as summarized in Table 1, Table 2, and Table 3, Table 4, Table 5, Table 6, and Table 7. While the Tables describe various formulas with respect to the parent ECM macromolecules detected therein, it is understood that the composition may include the matrikines, or fragmented peptides, derived from the corresponding parent ECM macromolecules. However, these compositions are exemplary in nature and the matrikine profiles may vary therefrom as to any number of components. For example, the composition may vary from the described concentration values and/or ranges by about 10%, about 20%, about 30%, greater than 30%, or individual values or ranges therebetween.

In some embodiments, the deconstructed matrisome comprises collagen. In some embodiments, the deconstructed matrisome comprises fragmented collagen. In some embodiments, the deconstructed matrisome comprises collagen in an amount from about 400 μg/mL to about 9700 μg/mL. In some embodiments, the deconstructed matrisome comprises collagen in an amount from about 2900 μg/mL to about 3800 μg/mL. In some embodiments, the deconstructed matrisome comprises collagen in an amount from about 7500 μg/mL to about 9500 μg/mL. In some embodiments, the deconstructed matrisome comprises collagen from about 1100 μg/mL to about 1300 μg/mL. In some embodiments, the deconstructed matrisome comprises collagen from about 400 μg/mL to about 530 μg/mL. In some embodiments, the deconstructed matrisome comprises collagen from about 7000 μg/mL to about 9700 μg/mL. In some embodiments the collagen is collagen type IV, wherein the collagen type IV is in an amount of about 2 ng/mL to about 24 ng/mL. In some embodiments the collagen is collagen type IV, wherein the collagen type IV is in an amount of about 6 ng/mL to about 10 ng/mL. In some embodiments the collagen is collagen type IV, wherein the collagen type IV is in an amount of about 20 ng/mL to about 24 ng/mL. In some embodiments the collagen is collagen type IV, wherein the collagen type IV is in an amount of about 9 ng/mL to about 19 ng/mL. In some embodiments the collagen is collagen type IV, wherein the collagen type IV is in an amount of about 2 ng/mL to about 10 ng/mL. In some embodiments the collagen is collagen type IV, wherein the collagen type IV is in an amount of about 3 ng/mL to about 5 ng/mL.

In some embodiments, the deconstructed matrisome comprises elastin. In some embodiments, the deconstructed matrisome comprises fragmented elastin. In some embodiments, the deconstructed matrisome comprises elastin in an amount from about 40 μg/mL to about 3000 μg/mL. In some embodiments the elastin is in an amount from about 40 μg/mL to about 50 μg/mL. In some embodiment the elastin is in an amount from about 350 μg/mL to about 450 μg/mL. In some embodiments the elastin is in an amount from about 120 μg/mL to about 150 μg/mL. In some embodiments the elastin is in an amount from about 1700 μg/mL to about 3000 μg/mL.

In some embodiments, the deconstructed matrisome comprises glycosaminoglycan. In some embodiments, the deconstructed matrisome comprises fragmented glycosaminoglycan. In some embodiments, the deconstructed matrisome comprises glycosaminoglycan in an amount from about 3 μg/ml to about 170 μg/ml. in some embodiments, the glycosaminoglycan is in an amount from about 130 μg/mL to about 170 μg/mL. In some embodiments, the glycosaminoglycan is in an amount from about 10 μg/mL to about 20 μg/mL. In some embodiments, the glycosaminoglycan is in an amount from about 5 μg/mL to about 15 μg/mL. In some embodiments, the glycosaminoglycan is in an amount from about 3 μg/mL to about 5 μg/mL. In some embodiments, the glycosaminoglycan is in an amount from about 80 μg/mL to about 110 μg/mL.

TABLE 1
Partial list of components in Matrikynes Formula 1.
MATRIKYNES ™ FORMULA 1

| Protein Category | Description |
|---|---|
| Collagens | Type I, alpha 1 chain |
| | Type III, alpha 1 chain |
| | Type V, alpha 2 chain |
| Glycoproteins | Fibrillar collagen NC1 domain-containing protein |
| | Fibrillin 1 |
| | Microfibril associated protein 4 |
| Proteoglycan | Heparan sulfate proteoglycan 2 |
| Elastin | Elastin isoform |
| Structural proteins | Actin gamma 2 |
| | Filamin A |
| Growth factors | Latent transforming growth factor |
| | beta binding protein 4 |

TABLE 2
Partial list of components in Matrikynes Formula 2.
MATRIKYNES ™ FORMULA 2

| Protein Category | Description |
|---|---|
| Collagens | Type I, alpha 1 chain |
| | Type I, alpha 2 chain |
| | Type II, alpha 1 chain |
| | Type III, alpha 1 chain |
| | Type V, alpha 1 chain |
| | Type V, alpha 2 chain |
| | Type VI, alpha 2 chain |
| | Type VI, alpha 3 chain |
| | Type VIII, alpha 1 chain |
| | Type IX, alpha 2 chain |
| | Type XI, alpha 1 chain |
| | Type XI, alpha 2 chain |
| | Type XII, alpha 2 chain |
| | Type XIV, alpha 1 chain |
| Glycoproteins | Adipocyte enhancer binding protein 1 |
| | Alpha-2-Heremans-Schmid glycoprotein |
| | Biglycan |
| | Extracellular matrix protein 2 |
| | Fibrillin 1 |
| | Fibrinogen beta chain |
| | Fibrinogen gamma chain |
| | Fibronectin 1 |
| | Osteonectin |
| | Periostin |
| | Tenascin C |
| | Tenascin N |
| | Thrombospondin 1 |
| | Transforming growth factor beta induced |
| | Vitronectin |
| Proteoglycans | Aggrecan core protein |
| | Asporin |
| | Decorin |
| | Fibromodulin |
| | Heparan sulfate proteoglycan 2 |
| | Lumican |
| | Mimecan |
| | Osteoglycan |
| | Osteomodulin |
| | Proline/arginine-rich end leucine-rich repeat protein |
| Elastin | Elastin |
| Matrisome secreted | Albumin |
| factors | Annexin A2 |
| | Chitinase |
| | Collectin subfamily member 12 |
| | Creatine kinase B |
| | Olfactomedin |
| ECM regulators | Coagulation factor IX |
| | Coagulation factor X |
| | Inter-alpha (globulin) inhibitor H4 |
| | Prothrombin |
| | Serpin peptidase inhibitor clade F |
| Structural proteins | Actin gamma 2 |
| | Vimentin |

TABLE 3
Partial list of components in Matrikynes Formula 3.
MATRIKYNES ™ FORMULA 3

| Protein Category | Description |
|---|---|
| Collagens | Type I, alpha 1 chain |
| | Type I, alpha 2 chain |
| | Type II, alpha 1 chain |
| | Type III, alpha 1 chain |
| | Type IV, alpha 1 chain |
| | Type IV, alpha 2 chain |
| | Type V, alpha 1 chain |
| | Type V, alpha 2 chain |
| | Type VI, alpha 2 chain |
| | Type VI, alpha 3 chain |
| | Type VI, alpha 5 chain |
| | Type VIII, alpha 1 chain |
| | Type VIII, alpha 2 chain |
| Glycoproteins | Dermatopontin |
| | Fibrillin 1 |
| | Microfibril-associated protein 4 |
| | Periostin |
| Proteoglycans | Asporin |
| | Heparan sulfate proteoglycan 2 |
| Elastin | Elastin isoform |
| Matrisome secreted | Chitinase |
| factors | Collectin subfamily member |
| | Trefoil factor 1 |
| | Vasoactive intestinal peptide |
| ECM regulators | Hyaluronan binding protein 2 |
| Structural Proteins | Actin gamma 2 |
| | Myosin 11 |
| Growth Factors | Amphiregulin |
| | Basic fibroblast growth factor |
| | Bone morphogenic protein 4 |
| | Bone morphogenic protein 7 |
| | Epidermal growth factor |
| | Growth differentiation factor 15 |
| | Hepatocyte growth factor |
| | Insulin-like growth factor binding protein 3 |
| | Osteoprotegerin |

TABLE 4
Partial list of components in Matrikynes Formula 4.
MATRIKYNES ™ FORMULA 4

| Protein Category | Description |
|---|---|
| Collagens | Type I, alpha 1 chain |
| | Type I, alpha 2 chain |
| | Type II, alpha 1 chain |
| | Type III, alpha 1 chain |
| | Type IV, alpha 1 chain |
| | Type V, alpha 2 chain |
| | Type VI, alpha 3 chain |
| | Type VI, alpha 5 chain |
| Glycoproteins | EGF-containing fibulin-like |
| | extracellular matrix protein |
| | Fibrillin 1 |
| | Fibrillin 2 |
| | Laminin subunit gamma 1 |
| | Prostate stem cell antigen |
| | Saposin-B-Val |
| | Von Willebrand factor |
| Proteoglycans | Heparan sulfate proteoglycan |
| Elastin | Elastin isoform |
| Matrisome secreted | Chitinase |
| factors | Mucin 5AC |
| | Mucin 6 |
| | Serum albumin |
| | Trefoil factor 2 |
| ECM regulators | Granulin precursor |
| Structural proteins | Actin |
| | Keratin 1 |
| | Keratin 2 |
| | Keratin 9 |
| | Keratin 10 |
| | Myosin heavy chain 9 |
| | Tubulin beta chain |
| Growth Factors | Bone morphogenic protein 4 |
| | Fibroblast growth factor 2 |
| | Insulin-like growth factor binding protein 4 |
| | Macrophage colony-stimulating |
| | factor 1 receptor (CD115) |
| | Pro-epidermal growth factor |

TABLE 5
Partial list of components in Matrikynes Formula 5.
MATRIKYNES ™ FORMULA 5

| Protein Category | Description |
|---|---|
| Collagens | Type I, alpha 1 chain |
| | Type I, alpha 2 chain |
| | Type I, alpha 3 chain |
| | Type II, alpha 1 chain |
| | Type IV, alpha 1 chain |
| | Type IV, alpha 2 chain |
| | Type IV, alpha 3 chain |
| | Type IV, alpha 4 chain |
| | Type IV, alpha 5 chain |
| | Type V, alpha 1 chain |
| | Type V, alpha 2 chain |
| | Type VI, alpha 1 chain |
| | Type VI, alpha 2 chain |
| | Type VI, alpha 3 chain |
| | Type VIII, alpha 1 chain |
| | Type XVI, alpha 1 chain |
| | Type XXI, alpha 1 chain |
| Glycoproteins | Dermatopontin |
| | Fibulin 2 |
| | Laminin subunit alpha 3 |
| | Laminin subunit alpha 5 |
| | Laminin subunit beta 2 |
| | Laminin subunit gamma 1 |
| | Nidogen 1 |
| | Periostin |
| | Vitronectin |
| Proteoglycans | Biglycan |
| | Heparan sulfate proteoglycan core protein |
| Elastin | Elastin isoform |
| Matrisome secreted | Hornerin |
| factors | |
| ECM regulators | Alpha 1 antitrypsin |
| | Cathepsin G |
| | Desmoplakin |
| | Junction plakoglobin |
| | Serum albumin precursor |
| | Metalloproteinase Inhibitor 3 |
| Structural proteins | Keratin 1 |
| | Keratin 2 |
| | Keratin 5 |
| | Keratin 9 |
| | Keratin 10 |
| | Keratin 14 |
| Growth Factors | Basic fibroblast growth factor |
| | Brain-derived neurotrophic factor |
| | Endocrine gland-derived vascular |
| | endothelial growth factor |
| | Epidermal growth factor receptor |
| | Growth differentiation factor 15 |
| | Hepatocyte growth factor |
| | Insulin-like growth factor binding protein 1 |
| | Insulin-like growth factor binding protein 6 |
| | Osteoprotegerin |
| | Platelet-derived growth factor AA |
| | Vascular endothelial growth factor |

TABLE 6
Quantification of extracellular matrix components
in five Matrikynes ™ Formulas.

| ECM Component | Formula 1 | Formula 2 | Formula 3 | Formula 4 | Formula 5 |
|---|---|---|---|---|---|
| Collagen type IV | 6-10 | 20-24 | 9-19 | 2-10 | 3-5 |
| (ng/mL) | | | | | |

TABLE 7
Major matricryptins/matrikines released from human extracellular parent proteins
Matrikines Released from Human ECM Proteins

| | | Matrikine |
|---|---|---|
| | Matrikine | Molecular Mass |
| Parent Protein | Common Name | (kDa) |
| Aggrecan core protein + | Metastatin (hyaluronan-binding | 85 + 38 |
| Proteoglycan link protein | complex) | |
| Collagen IV alpha 1 chain | Arresten | 26 |
| Collagen IV alpha 2 chain | Canstatin | 24 |
| Collagen IV alpha 3 chain | Tumstatin | 27 |
| Collagen IV alpha 4 chain | Tetrastatin | 2.1-25 |
| Collagen IV alpha 5 chain | Pentastatin 1 | 2.5 |
| | Pentastatin 2 | 2.4 |
| | Pentastatin 3 | 2.1 |
| | Lamstatin | 25 |
| Collagen IV alpha 6 chain | Hexastatin 1 | 2 |
| | Heastatin 2 | 2.5 |
| Collagen VI alpha 6 chain | Endotrophin | 5.8 |
| | (collagen-like 5 domain) | |
| Collagen XIII alpha 1 chain | Ectodomain of collagen XII | 240 |
| Collagen XV alpha 1 chain | Restin 1 | 21 |
| | Restin 2 | 20 |
| | Restin 3 | 19.4 |
| | Restin 4 | 18.4 |
| Collagen XVII alpha 1 chain | Ectodomain of collagen XVII | 120 |
| Collagen XVIII alpha 1 chain | Endostatin | 21 |
| | Neostatin 7 | 28 |
| | Neostatin 14 | 23 |
| Collagen XIX alpha 1 chain | NC1 domain of collagen XIX | 2.2 |
| Collagen XXIII alpha 1 chain | Ectodomain of collagen XXIII | 180 |
| Collagen XXV alpha 1 chain | Ectodomain of collagen XXV | 158 |
| | (collagen-like Alzheimer amyloid | |
| | plaque component) | |
| Fibronectin | Anastellin | 10 |
| | Fibstatin | 29 |
| Laminins | Laminin peptides | |
| MMP-2 | PEX | 24 |
| Perlecan | Endorepellin | 85 |
| Procollagen C-proteinase | CUB1CUB2 domain | 30 |
| enhancer 1 | | |
| Tenascin-C | Ten1/2 | 10 |
| | Ten11/12/13 | 3.4 |
| | Ten14 | |
| Syndecan-1 | Ectodomain of syndecan-1 | 24 |
| Syndecan-2 | Ectodomain of syndecan-2 | 14 |
| Syndecan-3 | Ectodomain of syndecan-3 | 39 |
| Syndecan-4 | Ectodomain of syndecan-4 | 14 |
| Tropoelastin | Elastokines | 0.5-8 |

In some embodiments the composition comprises one or more of collagen, glycoprotein, proteoglycan, elastin, matrisome secreted factors, structural proteins, growth factors, and ECM regulators.

In some embodiments the composition comprises one or more fragments of collagen, glycoprotein, proteoglycan, elastin, matrisome secreted factors, structural proteins, growth factors, and ECM regulators.

In some embodiments the compositions comprises collagen or fragments of collagen, wherein the collagen is selected from one or more of collagen type I alpha 1 chain, collagen type I alpha 2 chain, collagen type I alpha 3 chain, collagen type II alpha 1 chain, collagen type III alpha 1 chain, collagen type IV alpha 1 chain, collagen type IV alpha 2 chain, collagen type IV alpha 3 chain, collagen type IV alpha 4 chain, collagen type IV alpha 5 chain, collagen type V alpha 1 chain, collagen type V alpha 2 chain, collagen type VI alpha 1 chain, collagen type VI alpha 2 chain, collagen type VI alpha 3 chain, collagen type VI alpha 5 chain, collagen type VIII alpha 1 chain, collagen type VIII alpha 2 chain, collagen type IX alpha 2 chain, collagen type XI alpha 1 chain, collagen type XI alpha 2 chain, collagen type XII alpha 2 chain, collagen type XIV alpha 1 chain, collagen type XVI alpha 1 chain, collagen type XXI alpha 1 chain.

In some embodiments the composition comprises glycoproteins or fragments of glycoproteins, wherein the glycoprotein is selected from one or more of fibrillar collagen NC1 domain-containing protein, fibrillin, fibrillin 1, fibrillin 2, fibulin 2, microfibril associated protein 4, adipocyte enhancer binding protein 1, alpha-2-Heremans-Schmid glycoprotein, biglycan, extracellular matrix protein 2, fibrinogen beta chain, fibrinogen gamma chain, fibronectin 1, osteonectin, periostin, tenascin C, tenascin N, thrombospondin 1, transforming growth factor beta induced, vitronectin, dermatopontin, EGF-containing fibulin-like extracellular matrix protein, laminin, subunit alpha 3, laminin subunite alpha 5, laminin subunit beta 2, laminin subunit gamma 1, prostate stem cell antigen, saposin-B-Val, von Willebrand factor, and nidogen 1.

In some embodiments the composition comprises proteoglycans or fragments of proteoglycans, wherein the proteoglycan is selected from one or more of heparan sulfate proteoglycan, heparan sulfate proteoglycan 2, heparan sulfate proteoglycan core protein, aggrecan core protein, asporin, decorin, fibromodulin, lumican, mimecan, osteoglycan, osteomodulin, proline/arginine-rich end leucine-rich repeat protein, and biglycan.

In some embodiments, the composition comprises elastin or fragments of elastin, wherein the elastin is elastin isoform.

In some embodiments, the composition comprises matrisome secreted factors or fragments of matrisome secreted factors, wherein the matrisome secreted factors are selected from one or more of albumin, serum albumin, annexin A2, chitinase, collectin subfamily member 12, creatine kinase B, olfactomedin, trefoil factor 1, trefoil factor 2, vasoactive intestinal peptide, mucin SAC, mucin 5, and hornerin.

In some embodiments, the composition comprises structural proteins or fragments of structural proteins, wherein the structural proteins are selected from one or more of actin, actin gamma 2, filamin A, keratin 1, keratin 2, keratin 5, keratin 9, keratin 10, keratin 14, myosin 11, myosin heavy chain 9, tubulin beta chain, and vimentin.

In some embodiments, the composition comprises growth factors or fragments of growth factors, wherein the growth factors are selected from one or more of latent transforming growth factor beta binding protein 4, amphiregulin, basic fibroblast growth factor, bone morphogenic protein 4, bone morphogenic protein 7, brain-derived neurotrophic factor, epidermal growth factor, epidermal growth factor receptor, pro-epidermal growth factor, endocrine gland-derived vascular endothelial growth factor, fibroblast growth factor 2, growth differentiation factor 15, hepatocyte growth factor, insulin-like growth factor binding protein 1, insulin-like growth factor binding protein 3, insulin-like growth factor binding protein 4, insulin-like growth factor binding protein 6, macrophage colony-stimulating factor 1 receptor (CD115), osteoprotegerin, platelet-derived growth factor AA, vascular endothelial growth factor.

In some embodiments, the composition comprises ECM regulators or fragments of ECM regulators, wherein the ECM regulators are selected from one or more of alpha 1 antitrypsin, cathepsin G, coagulation factor IX, coagulation factor X, desmoplakin, granulin precursor, hyaluronan binding protein 2, inter-alpha (globulin) inhibitor H4, junction plakoglobin, metalloproteinase inhibitor 2, prothrombin, serpin peptidase inhibitor Glade F, and serum albumin precursor.

In some embodiments, the deconstructed matrisome comprises one or more fragments of collagens, glycoproteins, proteoglycans, elastins, matrisome secreted factors, structural proteins, growth factors, and ECM regulators, wherein the one or more collagens comprise collagen type I alpha 1 chain, collagen type III alpha 1 chain, and collagen type V alpha 2 chain; wherein one or more glycoproteins comprise fibrillar collagen NC1 domain-containing protein, fibrillin 1, and microfibril associated protein 4; wherein the one or more proteoglycans comprise heparan sulfate proteoglycan 2; wherein the one or more elastins comprise elastin isoform; wherein the one or more structural proteins comprise actin gamma 2 and filamin A; and wherein the one or more growth factors comprise latent transforming growth factor beta binding protein 4.

In some embodiments, the deconstructed matrisome comprises one or more fragments of collagens, glycoproteins, proteoglycans, elastins, matrisome secreted factors, structural proteins, growth factors, and ECM regulators, wherein the one or more collagens comprise collagen type I alpha 1 chain, collagen type I alpha 2 chain, collagen type II alpha 1 chain, collagen type III alpha 1 chain, collagen type V alpha 1 chain, collagen type V alpha 2 chain, collagen type VI alpha 2 chain, collagen type VI alpha 3 chain, collagen type VIII alpha 1 chain, collagen type IX alpha 2 chain, collagen type XI alpha 1 chain, collagen type XI alpha 2 chain, collagen type XII alpha 2 chain, and collagen type XIV alpha 1 chain; wherein the one or more glycoproteins comprise fibrillin 1, adipocyte enhancer binding protein 1, alpha-2-Heremans-Schmid glycoprotein, biglycan, extracellular matrix protein 2, fibrinogen beta chain, fibrinogen gamma chain, fibronectin 1, osteonectin, periostin, tenascin C, tenascin N, thrombospondin 1, transforming growth factor beta induced, and vitronectin; wherein the one or more proteoglycans comprise heparan sulfate proteoglycan 2, aggrecan core protein, asporin, decorin, fibromodulin, lumican, mimecan, osteoglycan, osteomodulin, and proline/arginine-rich end leucine-rich repeat protein; wherein the one or more elastins comprise elastin; wherein the one or more mastrisome secreted factors comprise albumin, annexin A2, chitinase, collectin subfamily member 12, creatine kinase B, olfactomedin; wherein the one or more ECM regulators are coagulation factor IX, coagulation factor X, inter-alpha (globulin) inhibitor H4, prothrombin, and serpin peptidase inhibitor Glade F; and wherein the one or more structural proteins are actin gamma 2 and vimentin.

In some embodiments, the deconstructed matrisome comprises one or more fragments of collagens, glycoproteins, proteoglycans, elastins, matrisome secreted factors, structural proteins, growth factors, and ECM regulators, wherein the one or more collagens comprise collagen type I alpha 1 chain, collagen type I alpha 2 chain, collagen type II alpha 1 chain, collagen type III alpha 1 chain, collagen type IV alpha 1 chain, collagen type IV alpha 2 chain, collagen type V alpha 1 chain, collagen type V alpha 2 chain, collagen type VI alpha 2 chain, collagen type VI alpha 3 chain, collagen type VI alpha 5 chain, collagen type VIII alpha 1 chain, and collagen type VIII alpha 2 chain; wherein the one or more glycoproteins comprise dermatopontin, fibrillin 1, microfibril-associate protein 4, and periostin; wherein the one or more proteoglycans comprise asporin and heparan sulfate proteoglycan 2; wherein the one or more elastins comprise elastin isoform; wherein the one or more matrisome secreted factors comprise chitinase, collectin subfamily member, trefoil factor 1, and vasoactive intestinal peptide; wherein the one or more ECM regulators comprise hyaluronan binding protein 2; wherein the one or more structural proteins comprise actin gamma 2 and myosin 11; and wherein the one or more growth factors comprise amphiregulin, basic fibroblast growth factor, bone morphogenic protein 4, bone morphogenic protein 7, epidermal growth factor, growth differentiation factor 15, hepatocyte growth factor, insulin-like growth factor binding protein 3, and osteoprotegerin.

In some embodiments, the deconstructed matrisome comprises one or more fragments of collagens, glycoproteins, proteoglycans, elastins, matrisome secreted factors, structural proteins, growth factors, and ECM regulators, wherein the one or more collagens comprise collagen type I alpha 1 chain, collagen type I alpha 2 chain, collagen type II alpha 1 chain, collagen type III alpha 1 chain, collagen type IV alpha 1 chain, collagen type V alpha 2 chain, collagen type VI alpha 3 chain, collagen type VI alpha 5 chain; wherein the one or more glycoproteins comprise fibrillin 1, fibrillin 2, EGF-containing fibulin-like extracellular matrix protein, laminin subunit gamma 1, prostate stem cell antigen, saposin-B-Val, and von Willebrand factor; wherein the one or more proteoglycans comprise heparan sulfate proteoglycan; wherein the one or more elastins comprise elastin isoform; wherein the one or more matrisome secreted factors comprise chitinase, mucin SAC, mucin 6, serum albumin, and trefoil factor 2; wherein the one or more ECM regulators comprise granulin precursor; wherein the one or more structural proteins comprise actin, keratin 1, keratin 2, keratin 9, keratin 10, myosin heavy chain 9, and tubulin beta chain; and wherein the one or more growth factors comprise bone morphogenic protein 4, fibroblast growth factor 2, insulin-like growth factor binding protein 4, macrophage colony-stimulating factor 1 receptor (CD115), and pro-epidermal growth factor.

In some embodiments, the deconstructed matrisome comprises one or more fragments of collagens, glycoproteins, proteoglycans, elastins, matrisome secreted factors, structural proteins, growth factors, and ECM regulators, wherein the one or more collagens comprise collagen type I alpha 1 chain, collagen type I alpha 2 chain, collagen type I alpha 3 chain, collagen type II alpha 1 chain, collagen type IV alpha 1 chain, collagen type IV alpha 2 chain, collagen type IV alpha 3 chain, collagen type IV alpha 4 chain, collagen type IV alpha 5 chain, collagen type V alpha 1 chain, collagen type V alpha 2 chain, collagen type VI alpha 1 chain, collagen type VI alpha 2 chain, collagen type VI alpha 3 chain, collagen type VIII alpha 1 chain, collagen type XVI alpha 1 chain, and collagen type XXI alpha 1 chain; wherein the one or more glycoproteins comprise fibulin 2, periostin, vitronectin, dermatopontin, laminin subunit alpha 3, laminin subunite alpha 5, laminin subunit beta 2, laminin subunit gamma 1, and nidogen 1; wherein the one or more proteoglycans comprise biglycan and heparan sulfate proteoglycan core protein; wherein the one or more elastins comprise elastin isoform; wherein the one or more matrisome secreted factors comprise hornerin; wherein the one or more ECM regulators comprise alpha 1 antitrypsin, cathepsin G, desmoplakin, junction plakoglobin, serum albumin precursor, and metalloproteinase inhibitor 3; wherein the one or more structure proteins comprise keratin 1, keratin 2, keratin 5, keratin 9, keratin 10, and keratin 14; and wherein the one or more growth factors comprise basic fibroblast growth factor, brain-derived neurotrophic factor, epidermal growth factor receptor, endocrine gland-derived vascular endothelial growth factor, growth differentiation factor 15, hepatocyte growth factor, insulin-like growth factor binding protein 1, insulin-like growth factor binding protein 6, osteoprotegerin, platelet-derived growth factor AA, and vascular endothelial growth factor.

The resulting mixture of matrikines in the composition may signal and modulate unique bioactivity based on the specific mixture and concentrations thereof. For example, the matrikines may signal proliferation, migration, protease production, apoptosis, cell interaction, gene expression, and ECM remodeling, thereby promoting tissue repair and regeneration. In some embodiment, the deconstructed matrisomes comprising enzymatically fragmented matrikines of the present application emulate biological signaling related to tissue injury and can trigger an immune response to promote healing. For example, the fragmentation of the matrisome may emulate a tissue injury, thereby releasing matrikines and triggering signaling that emulates a natural biological response to tissue injury. Accordingly, the deconstructed matrisome may trigger an immune response and/or promote healing by activating signaling and cell interactions that emulate the natural biological response to a tissue injury. In some embodiments, where the composition is applied to a tissue that does not have a current or recent injury, the deconstructed matrisome may nonetheless trigger an immune response and/or promote healing therein.

The composition described herein may be unique from TS-ECM or matrikine compositions produced by various conventional methods by the inclusion of these various components. While conventional methods utilize intact sheets, slices, or sections, of ECM scaffold from natural tissue for cell culturing, the scaffold alone may have several drawbacks. While intact scaffold may be used as a covering for skin or other surfaces, it may not penetrate and absorb through the surface due to the ECM format as well as the fragment size.

Additionally, matrikines often display cryptic bioactivities not manifested by the native, full-length parent proteins or molecules. While some matrikines may be active in the full-length form, many matrikines are only active after being fragmented as described herein. In some cases, the matrikines may promote opposite activities as the parent protein. Thus, intact sheets may not present the same signaling and modulation of bioactivity as the fragmented child peptides. Exemplary active matrikines which differ in this bioactivities with respect to their parent macromolecules are listed in Table 7 (Ricard-Blum and Salza, “Matricryptins and matrikines: biologically active fragments of the extracellular matrix,” Exper. Dermat., 2014, 23, 457-463, incorporated by reference herein in its entirely).

Furthermore, intact sheets of scaffold may lack several components found only in the ECF and/or the greater matrisome. Furthermore, the concentrations of various components in the scaffold alone may differ from the concentrations of the same components in the whole tissue (i.e., due to the differing composition of the greater matrisome). The matrikines described herein may be produced from TS-ECM through processing ECM scaffold and tissue in a manner that does not remove or compromise components of the extracellular environment beyond the scaffold. Therefore, the composition described herein may signal and/or modulate bioactivity in a manner unique from an intact sheet or covering of ECM scaffold from the same tissue source.

In contrast, the compositions described herein may comprise enzymatically fragmented deconstructed matrisome derived from ECM, thereby activating additional matrikine bioactivity and having reduced size capable of absorbing deeper into the dermal layers of skin. The isolated acellular extracellular matrix may be processed into fragments by digestion with enzymes such as proteases as would be apparent to a person having an ordinary level of skill in the art to produce fragments sized to be absorbed through one or more dermal layers, e.g., absorption into the epidermis, absorption through the epidermis and into the dermis, and/or absorption through the dermis and into the hypodermis. For example, deconstructed matrisome fragments having a size less than about 500 Da may be configured for absorption into the epidermis and the dermis. However, the fragments in the compositions described herein may have a variety of sizes, such as about 250 kDa, about 200 kDa, about 150 kDa, about 100 kDa, about 50 kDa, about 25 kDa, about 10 kDa, about 5 kDa, about 1 kDa, about 500 Da, about 250 Da, about 100 Da, about 50 Da, less than about Da, and/or individual values or ranges therebetween.

The matrisome may be deconstructed through one or more fragmentation processes as described herein, which may include chemical fragmentation such as enzymatic digestion (e.g., by proteases) and/or physical fragmentation by application of a force to the source material (i.e., biological tissue). It should be understood that the matrisome may be fragmented to a greater degree than conventional processes, such as fragmentation for the purposes of producing a ECM-based cell culture substrate. Accordingly, by applying a variety of fragmentation process as described, the deconstructed matrisome may comprise fragmented peptides as small as two or more amino acids. While certain applications such as cell culture only require a limited degree of fragmentation, the subject matter disclosed herein provides for a greater degree of fragmentation resulting in fragments sized and configured for absorption into one or more dermal layers. Furthermore, the greater degree of fragmentation may emulate biological signaling related to tissue injury. For example, the fragmentation of the matrisome by the methods described herein may emulate a tissue injury, thereby releasing matrikines and triggering signaling that emulates a natural biological response to tissue injury. Accordingly, the deconstructed matrisome may comprise fragments that are capable of triggering an immune response and/or promoting healing by activating signaling and cell interactions that emulate the natural biological response to a tissue injury. In some embodiments, where the composition is applied to a tissue that does not have a current or recent injury, the deconstructed matrisome may nonetheless be capable of triggering an immune response and/or promoting healing therein.

Furthermore, the deconstructed matrisome may have a specified composition that emulates the matrisomes found in a specific native tissue and/or combinations thereof. As such, the composition of each deconstructed matrisome may vary. It should be understood that the deconstructed matrisome includes a “complete matrisome,” i.e., a full complement of naturally defined ECM components from one or more biological tissues to provide a complete set of fragmented macromolecules for the purposes of promoting cell interactions and signaling.

Each deconstructed matrisome may comprise a different combination of proteoglycans, collagens, elastins, multiadhesive proteins, hyaluronic acid, CAMs, and additional components. Each of these components may have subtypes, the presence of each of which may vary from one deconstructed matrisome to another deconstructed matrisome. Each deconstructed matrisome may be characterized by the presence or absence of one or more components. Further, the concentration of each component may vary from one deconstructed matrisome to another deconstructed matrisome. These variations result in each deconstructed matrisome having unique physical characteristics, such as architecture and stiffness, and unique cell interaction characteristics, such as gene expression, ECM remodeling, and cell proliferation.

Further, the fragmented deconstructed matrisome may be decellularized, isolated and processed in a manner that does not remove or compromise components of the extracellular environment beyond the scaffold. Therefore, the ECM substrates described herein include components beyond that which is found in ECM scaffold in vivo, thereby more accurately emulating the in vivo extracellular environment of the tissue.

In some embodiments, the enzymatically fragmented deconstructed matrisome may comprise additional components beyond those present in the native extracellular matrix. In some embodiments, the enzymatically fragmented deconstructed matrisome may include components found in the extracellular fluid of a specific tissue. For example, a component present in extracellular fluid of skin tissue may not be present in the ECM scaffold thereof and may thus be added to the ECM to further emulate the niche environment. In some embodiments, the enzymatically fragmented deconstructed matrisome may include one or more of amino acids, glucose, salts, vitamins, carbohydrates, proteins, peptides, trace elements, other nutrients, extracts, additives, gases, or organic compounds. Additional components for the proper growth, repair, and regeneration of skin as would be known to one having an ordinary level of skill in the art are also contemplated herein.

As described herein, the matrikines may be configured to support tissue regeneration and healing. Further, the matrikines may be configured to facilitate growth and proliferation of the human skin fibroblasts in a manner consistent with tissue healing. Accordingly, the matrikines may induce gene expression, growth factor secretion, and other characteristics in a manner consistent with tissue healing. However, the matrikines may be configured to support a variety of additional cell types found in the skin, i.e., native cells.

In some embodiments, the topical formulation may have a pH of less than about 6.0. In some embodiments, the topical formulation has a pH of less than about 5.5. In some embodiments, the topical formulation has a pH selected from the group consisting of about 4.0 to about 6.0, about 4.5 to about 5.0, and about 4.4 to about 4.7. The topical formulation may be configured to lower a pH of a skin surface to reduce or eliminate pathogen presence or growth.

The topical composition may take a variety of forms. In some embodiments, the composition is formulated in a form selected from the group consisting of solution, fluid, emulsion, suspension, solid, semi-solid, jelly, paste, gel, hydrogel, ointment, lotion, serum, cream, foam, mousse, liquid, spray, suspension, dispersion, powder, aerosol, film, or transdermal patches formulated as a liquid, cream, ointment, gel, aerosol, neck cream, neck lotion, body lotion, body cream, face lotion, face cream, eye lash treatment, hair moisturizer, hair conditioner, cellulite treatment, nail conditioner, gel, emulsion, silicone gel, water gel, oil-in-water emulsion, or water-in-oil emulsion. I

In some embodiments, the topical composition comprises about 0.01% by weight to about 25% by weight of deconstructed matrikines. In some embodiments, the topical composition comprises about 0.1% by weight to about 15% by weight of deconstructed matrikines. In some embodiments, the topical composition comprises about by weight to about 2.5% by weight of deconstructed matrikines. In some embodiments, the topical composition comprises deconstructed matrikines in an amount from about 0.01% to about 15% by weight, about 0.01% to about 10% by weight, about 0.01% to about 7.5% by weight, about 0.01% to about 5% by weight, about 0.01% to about 2.5% by weight, about to about 1% by weight, about 0.01% to about 0.9% by weight, about 0.01% to about by weight, about 0.01% to about 0.7% by weight, about 0.01% to about 0.6% by weight, about 0.01% to about 0.5% by weight, about 0.01% to about 0.4% by weight, about to about 0.3% by weight, about 0.01% to about 0.2% by weight, about 0.01% to about by weight, about 0.01% to about 0.075% by weight, about 0.01% to about 0.05% by weight, about 0.01% to about 0.025% by weight, or individual values or ranges therebetween.

Liquid dosage forms for topical administration may comprise diluents such as, for example, alcohols, glycols, oils, water, and the like. Such compositions may also include wetting agents or emulsifiers.

A cream can be a water-in-oil (w/o) emulsion in which an aqueous phase is dispersed in an oil phase, or an oil-in-water (o/w) emulsion in which an oil is dispersed within an aqueous base. An ointment generally refers to a more viscous oil-in-water cream. Traditional ointment bases (i.e. carrier) include hydrocarbons (petrolatum, beeswax, etc.) vegetable oils, fatty alcohols (cholesterol, lanolin, wool alcohol, stearyl alcohol, etc.) or silicones. Insoluble solids such as starch, zinc oxide, calcium carbonate, or talc can also be used in ointments and creams. Gel forms of the compositions described above can be formed by the entrapment of large amounts of aqueous or aqueous-alcoholic liquids in a network of polymers or of colloidal solid particles. Such polymers or colloids (gelling or thickening agents) are typically present at concentrations of less than 10% w/w and include carboxymethyl cellulose, hydroxypropylmethyl cellulose, hydroxyethyl cellulose, methyl cellulose, sodium alginate, alginic acid, pectin, tragacanth, carrageen, agar, clays, aluminum silicate, carbomers, and the like.

In aerosols the composition is dissolved in a propellant such as dichlorodifluoromethane, trichlorofluoromethane, dichlorotetrafluoroethane, carbon dioxide, or other suitable gas, and a co-solvent such ethanol, acetone, hexadecyl alcohol, and combinations thereof.

Hydrogels are typically prepared by cross-linking various monomers and/or polymers to provide a three-dimensional polymer network. Non-limiting examples of polymers include, polyoxyethylene-polypropylene block copolymers, ionic poly saccharides, such as chitosan or sodium alginate, cellulose, and biodegradable polymers, such as poly-lactides (PLA) and poly-glycolides (PGA), butylene succinate (PBS), polyhydroxyalkanoate (PHA), polycaprolactone acid lactone (PCL), polyhydroxybutyrate (PHB), glycolic amyl (PHV), PHB and PHV copolymer (PHBV), and poly lactic acid (PLA)-polyethylene glycol (PEG) copolymers (PLEG).

The transdermal patches can be in any conventional form such as, for example, a strip, a gauze, a film, and the like. Patch material may be nonwoven or woven (e.g., gauze dressing). Layers may also be laminated during processing. It may be nonocclusive or occlusive, but the latter is preferred for backing layers. The patch is preferably hermetically sealed for storage (e.g., foil packaging). The patch can be held onto the skin and components of the patch can be held together using various adhesives. For example, the transdermal patch can be in the form of a Band-Aid type device, or it may be packaged in a small metal or plastic “cup”, which is strapped onto the appropriate site using an adhesive, tape, or an outer fabric or leather strap, similar to that worn as part of a watch. The entire patch may be disposable or may be refillable. In embodiments, the formulation can be formulated with a latex polymer, wherein the formulation is applied to the skin and forms an occlusive film.

In some embodiments, the formulations disclosed herein can be coated on bandages, mixed with bio adhesives, or included in dressings.

In some embodiments, the formulations disclosed herein can be used in combination with a cosmetic device.

In some embodiments, the formulations disclosed herein can be used in combination with a patch.

In some embodiments, the formulation is part of an anti-aging regimen. In some embodiments, the formulation is part of regimen for after sun care. In some embodiments, the formulation is part of a photoprotective regimen. In some embodiments, the photoprotective regimen is a sunblock regimen or a sunscreen. In some embodiments, the formulation is part of regimen for skin lightening. In some embodiments, the formulation is part of regimen for skin brightening. In some embodiments, the formulation is part of regimen for acne treatment. In some embodiments, the formulation is part of regimen for inflammation treatment. In some embodiments, the formulation is part of a color cosmetic regimen. In some embodiments, the formulation is part of a hair treatment regimen. In some embodiments, the antioxidant composition is part of a scalp treatment regimen.

In some embodiments, the topical formulation further comprises a solvent. In some embodiments, the solvent is selected from the group consisting of pentylene glycol, butylene glycol, water, glycols, propylene glycol, isopropylene glycol, coco-caprylate/caprate, 1,2-hexanediol, and combinations thereof.

In some embodiments, the topical formulation further comprises a pharmaceutical additive, a cosmetic additive, an additional agent, water, or combinations thereof. In some embodiments, the topical formulation further comprises both a pharmaceutical additive and a cosmetic additive. In some embodiments, the pharmaceutical additive, the cosmetic additive, the additional additives, or combination thereof are in a total amount of at least about 12 wt %. In some embodiments, the pharmaceutical additive, the cosmetic additive, the additional additive, or combination thereof are in a total amount selected from the group consisting of about 12 wt % to about 90 wt %, about 12 wt % to about 85 wt %, about 12 wt % to about 80 wt %, about 15 wt % to about 90 wt %, about 15 wt % to about 85 wt %, about 15 wt % to about 80 wt %, about 15 wt % to about 75 wt %, about 20 wt % to about 70 wt %, about wt % to about 65 wt %, about 30 wt % to about 60 wt %, about 35 wt % to about 55 wt %, and about 40 wt % to about 50 wt %. In some embodiments, the topical formulation may comprise water in a percentage of at least about 68 wt %. In some embodiments, the topical formulation may comprise water in a percentage selected from the group consisting of about 68 wt % to about 90 wt %, about 70 wt % to about 88 wt %, about 72 wt % to about 86 wt %, about 74 wt % to about 84 wt %, about 76 wt % to about 82 wt %, and about 78 wt % to about wt %.

In some embodiments, the pharmaceutical additive is selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plastizers, carriers, excipients, and combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of embodiments.

In some embodiments, the cosmetic additive is selected from the group consisting of vitamins, cosmetic peptides, oil control agents, sensation modifying agents, skin lightening agents, hydrating formulations, sunblock agents, a compounds that absorbs or reflects UV photons, other skin care agents, and combinations thereof.

In some embodiments, the additional additive is selected from the group consisting of hydroxyacetophenone, sodium phytate, caprylic/capric triglyceride, sodium acrylates copolymer, octyldodecanol, octyldodecyl xyloside, PEG-30 dipolyhydroxystearate, Jojoba esters, Helianthus annuus (sunflower) seed wax, Acacia decurrens flower wax, polyglycerin-3, acrylamide/sodium acryloyldimethyl taurate copolymer, isohexadecane, polysorbate 80, cyclopentasiloxane, dimethicone/vinyl dimethicone crosspolymer, ethylhexylglycerin, and combinations thereof.

In some embodiments, the topical formulation may further comprise abrasives, antiacne agents, antidandruff agents, antifungal agents, antimicrobial agents, antioxidants, toners, moisturizers, skin conditioning agents, humectants, emollients, occlusive agents, skin bleaching or lightening agents, proteins, cleaners, hair conditioners, and the like.

Abrasives may be used to remove unwanted skin such as dead skin cells and calluses. In some embodiments, the abrasive is selected from the group consisting of alumina, aluminum silicate, apricot seed powder, attapulgite, avocado powder, bamboo powder, barley flour, bentonite, calcium carbonate, calcium phosphate, calcium pyrophosphate, calcium sulfate, chalk, chitin, coconut shell powder, colloidal oatmeal, comfrey leaf powder, corn cob meal or powder, corn flour, corn meal, corn starch, diamond powder, diatomaceous earth, dicalcium phosphate, dicalcium phosphate dehydrate, egg shell powder, Fuller's earth, hydrated silica, hydroxyapatite, kaolin, kiwi seed, lauryl acrylate polymers, loess, magnesium potassium fluorosilicate, magnesium trisilicate, microcrystalline cellulose, montmorillonite, Moroccan lava clay, oat bran, oat flour, oatmeal, oyster shell powder, peach pit powder, peanut flour, pecan shell powder, polyethylene, pumice, raspberry seed, rice bran, rye flour, sand, silica, sodium bicarbonate, sodium hydroxypropyl starch phosphate, sodium magnesium fluorosilicate, sodium silicoaluminate, soybean flour, sweet almond meal, talc, tin oxide, tricalcium phosphate, walnut shell powder, wheat bran, wheat flour, wheat powder, wheat starch, wood powder, zirconium silicate, and derivatives and combinations thereof.

Antiacne agents may be used to treat blemishes, pimples, blackheads, and whiteheads. In some embodiments, the antiacne agent is selected from the group consisting of salicylic acid, benzoyl peroxide, carbamide peroxide, glycolic acid, retinal, retinol, retinaldehyde, vitamin A, vitamin A derivative, azelaic acid, or sulfur, and their derivatives and combinations thereof.

Antidandruff agents may be used to treat dandruff, seborrheic dermatitis, or psoriasis. In some embodiments, the antidandruff agent is selected from the group consisting of coal tar, salicylic acid, selenium sulfide, sulfur, zinc pyrithione, and their derivatives and combinations thereof.

Antifungal agents include agents that inhibit the growth and reproduction of fungal cells or decreases the number of fungi present. In some embodiments, the antifungal agent is selected from the group consisting of calcium undecylenate, ketoconazol, povidone-iodine (PVP-iodine), tea tree oil, undecylenic acid, zinc undecylenate, and their derivatives and combinations thereof.

Antimicrobial agents include agents that kill microorganisms, prevent or inhibit microorganism growth and reproduction, or agents that help prevent infection in minor cuts, scrapes, and burns. In some embodiments, the antimicrobial agent is selected from the group consisting of lower chain (C1-C₄) alcohols, quaternary ammonium compounds such as benzalkonium chloride and benzethonium chloride, clindamycin, methylbenzethonium chloride, hydrogen peroxide, Oligopeptide-10, phenols, tea tree oil, triclosan, povidone-iodine (PVP-Iodine), and their derivatives and combinations thereof.

Antioxidants include agents that are characterized as free radical scavengers and help reverse skin damage caused by free radicals. In some embodiments, the antioxidant is selected from the group consisting of acetyl cysteine, alpha lipoic acid, arbutin, ascorbic acid (vitamin C), ascorbic acid polypeptide, ascorbyl dipalmitate, ascorbyl methylsilanol pectinate, ascorbyl palmitate, ascorbyl stearate, BHA, BHT, t-butyl hydroquinone, caffeic acid, Camellia sinensis oil, carotenoids, chitosan ascorbate, chitosan glycolate, chitosan salicylate, chlorogenic acids, CoQ10, cortisen, cysteine, cysteine HCl, decyl mercaptomethylimidazole, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, diosmine, disodium ascorbyl sulfate, disodium rutinyl disulfate, distearyl thiodipropionate, ditridecyl thiodipropionate, dodecyl gallate, dunaliella salina extract, erythorbic acid, ethyl ferulate, ferulic acid, hydroquinone, p-hydroxyanisole, hydroxylamine HCl, hydroxylamine sulfate, hydroxytyrosol, isooctyl thioglycolate, isoquercitrin, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycloic acid, phloroglucinol, potassium ascorbyl tocopheryl phosphate, potassium sulfite, propyl gallate, resveratrol, rosmarinic acid, rutin, sirtunis, sodium ascorbate, sodium ascorbyl/cholesteryl phosphate, sodium bisulfite, sodium erythorbate, sodium metabisulfite, sodium sulfite, sodium thioglycolate, sorbityl furfural, tea tree oil, tetrahexyldecyl ascorbate, tetrahydrodiferuloylmethane, thiodiglycol, thiodiglycolamide, thiodiglycolic acid, thioglycolic acid, thiolactic acid, thiosalicylic acid, thiotaurine, tocophereth derivatives, tocopherol (vitamin E), tocophersolan, tocopheryl acetate, tocopheryl linoleate, tocopherol linoleate/oleate, tocopheryl nicotinate, tocopheryl succinate, tocoquinone, o-tolyl biguanide, tri(nonylphenyl)phosphate, ubiquinone, vitamin D, zinc dibutyldithiocarbamate, and their derivatives and combinations thereof.

Toners include agents that create a tightening or tingling sensation on skin. In some embodiments, the toner is selected from the group consisting of ammonium alum, calcium chloride, calcium lactate, dimethyl MEA, gallic acid, lens esculenta (lentil) seed extract, potassium alum, sodium alum, sodium aluminum chlorohydroxy lactate, sodium aluminum lactate, tannic acid, tioxolone, tranexamic acid, zinc acetate, zinc chloride, zinc lactate, zinc phenolsulfonate, zinc sulfate, zirconium chlorohydrate, witch hazel, alcohol derivatives such as denatured alcohol and SD alcohol, aluminum derivatives such as aluminum acetate, aluminum bromohydrate, aluminum chloride, aluminum chlorohydrex, aluminum citrate, aluminum diacetate, aluminum dichlorohydrate, aluminum dichlorohydrex, aluminum glycinate, aluminum lactate, aluminum phenolsulfonate, aluminum sesquichlorohydrate, aluminum sesquichlorohydrex, and aluminum sulfate, aluminum zirconium derivatives such as aluminum zirconium octachlorohydrex, aluminum zirconium pentachlorohydrate, aluminum zirconium pentachlorohydrex, aluminum zirconium tetrachlorohydrate, aluminum zirconium tetrachlorohydrex, aluminum zirconium trichlorhydrate, and aluminum zirconium trichlorohydrex, and their derivatives and combinations thereof.

Skin conditioning agents or moisturizers can be classified into different groups such as emollients, humectants, and occlusive agents. Emollients include agents that remain on the upper layers of skin and act as lubricants and improve appearance. In some embodiments, the emollient is selected from the group consisting of petrolatum, petrolatum plus volatile silicones, cold cream (USP), hydrophilic ointment (USP), lanolin, glycerides, fruit oils, nut oils, vegetable oils, isopropyl palmitate, dimethicones, methicone, cyclomethicone, dormin, fatty acids, myristate derivatives like butyl myristate and myristyl myristate, oleate derivates, C1-C4 glycols, fatty acid glycols, glycol esters, glycerine, glycerols, paraffin, rapeseed oil, long chain alcohols, olive oil, jojoba oil, castor oil, and their derivatives and combinations thereof. Humectants include agents that increase the water content of the top layer of skin. In some embodiments, the humectant is selected from the group consisting of allatoin, agarose, arginine, benzyl hyaluronate, chitosan, copper, corn glycerides, gluconolactone, lactic acid, lactobionic acid, lactose, lysine, kombucha, maltitol, maltose, mannitol, propylene glycol, butylene glycol, pentylene glycol, propanediol, sodium aspartate, fructose, honey, glycerin, diglycerin, betaine, diols, hydroxyethyl urea, 1,2-hexanediol, D-ribose, glucose, sorbitol, dextrose, urea, 2-Pyrrolidone-5-Carboxylic Acid and related salts, sea salt, inorganic salts of citric acid, inorganic salts of lactic acid, ectoin, glycolic acid, and their derivatives and combinations thereof. Occlusive agents slow the evaporation of water from skin. In some embodiments, the occlusive agent is selected from the group consisting of petrolatum, shea butter, dimethicones, plant and animal oils such as avocado, canola, cod liver, and corn, mineral oil, olive oil, soybean oil, lanolin, glycerides, beeswax, triglycerides, long chain fatty alcohols, coco butter, coconut oil, jojoba oil, propylene glycol and their derivatives and combinations thereof.

In addition to skin conditioning agents that provide a moisturizing benefit, there are other skin conditioning agents that improve the appearance of skin. In some embodiments, the skin conditioning agent is selected from the group consisting of cholesterol, cystine, hyaluronic acid, keratin, egg yolk, glycine, gluconolactone, lactic acid, lactobionic acid, panthenol, retinol, salicylic acid, vegetable oil, proteins, vitamins, bisabolol, ceramide, coenzyme A, lecithin and their derivatives and combinations thereof.

Skin bleaching or lightening agents include agents that lighten pigment in skin. The preferred skin bleaching agent is hydroquinone. In some embodiments, the brightener is selected from the group consisting of azelaic acid, bearberry, deoxyarbuten, Glycyrrhiza glabra (Licorice) root extract, kojic acid, peat extract, and their derivatives and combinations thereof.

Hair conditioners include agents that enhance the appearance and feel of hair by improving a property like gloss, texture, or body. In some embodiments, the hair conditions is selected from the group consisting of lanolin, silicone, dimethicone, proteins such as amino acids, collagen, and keratin, vitamins, betaine surfactants, amine oxide surfactants, ceramide, fatty acids, eggs, milk, natural plant and animal oils, mineral oil, olive oil, polyquaternium, and their derivatives and combinations thereof.

Proteins include animal, plant, fungi, yeast, and bacteria proteins that have skin health benefits. In some embodiments, the protein is selected from the group consisting of collagen, keratin, soy protein, wheat protein, bean palmitate, ascorbic acid polypeptide, the amino acids, casein, cholecalciferol polypeptide, rice protein, silk protein, gluten protein, lysine, acetyl glucosamine, actin, actizyme, albumen, conchiorin protein, corn protein, egg protein, elastin, fibronectin, gadidae protein, hemoglobin, hexapeptide-21, lactalalbumin, lupine protein, maple sycamore protein, milk protein, myristoyl pentapeptide-8, myristoyl tetrapeptide-8, oat protein, oligopeptide 10, palmitoyl hexapeptide-14, palmitoyl oligopeptide, palmitoyl tetrapeptide-7, pea protein, potato protein, reticulin, rice bran protein, serum protein, sweet almond protein, tetrapeptide-16, vegetable protein, yeast protein, palmitoyl oligopeptide, pantothenic acid polypeptides, milk solids, sericin, albumen, amylase, amyloglucosidase, arginine, bromelain, catalase, gelatin, zein, crystallins, cytochrome C, deoxyribonuclease, gliadin, glucose oxidase, glycoproteins, lactoferrin, lactoglubulin, lactoperoxidase, lipase, nisin, oxido reductases, papain, pepsin, subtilisin, sutilains, and their derivatives and combinations thereof.

Cleansers include agents that are used for cleaning the skin and hair by solubilizing oil and suspending soils. Cleansers may be foaming or non-foaming. Exemplary cleaners are typically a surfactant and can be characterized as nonionic, anionic, or zwitterionic. In some embodiments, the cleanser is selected from the group consisting of taurates, sulfates, sulfonates, carboxylates, sulfosuccinates, sarcosinates, zwitterionic betaines, fatty acid and fatty alcohol derivatives, and alkylpolyglucoside and amine oxide surfactants. In some embodiments, the cleansers may be combined with some abrasives such as clays and sulfurs to provide light exfoliation.

In some embodiments, the topical formulation further comprises a gelling agent. In some embodiments, the gelling agent is a water phase gelling agent. In some embodiments, the water phase gelling agent is selected from the group consisting of xanthan gum, gellan gum, carrageenan, biosaccharide gum-I, sclerotium gum, pectin, pullulan, guar gum, gum arabic, chondroitin, sulfate, alginic acid, sodium hyaluronate, hydrolyzed hyaluronic acid sodium polyglutamate, chitin, chitosan, starch, and combinations thereof. In some embodiments, the gelling agent is xanthan gum.

In some embodiments, the topical formulation may further comprise an oil control agent. Oil control agents are compounds useful for regulating the production of skin oil, or sebum, and for improving the appearance of oily skin. In some embodiments, the oil control agent is selected from the group consisting of salicylic acid, dehydroacetic acid, benzoyl peroxide, vitamin B3 (for example, niacinamide), and the like, their isomers, esters, salts and derivatives, and combinations thereof.

In some embodiments, the topical formulation may further comprise other skin care agents selected from the group consisting of retinol, steroids, sunblock, salicylate, minocycline, antifungals, peptides, antibodies, lidocaine, and the like and combinations thereof. In some embodiments, other skin care agents include N-acyl amino acid compounds comprising, for example, N-acyl phenylalanine, N-acyl tyrosine, and the like, their isomers, comprising their D and L isomers, salts, derivatives, and mixtures thereof. An example of a suitable N-acyl amino acid is N-undecylenoyl-L-phenylalanine is commercially available under the tradename SEPIWHITE®. Other skin active agents include, but are not limited to, Lavandox, Thallasine 2, Argireline NP, Gatuline In-Tense and Gatuline Expression, Myoxinol LS 9736, Syn-ake, and Instensyl®, Sesaflash™, N-acetyl D-glucosamine, panthenol (for example, DL panthenol available from Alps Pharmaceutical Inc.), tocopheryl nicotinate, benzoyl peroxide, 3-hydroxy benzoic acid, flavonoids (for example, flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (for example, retinyl propionate), phytic acid, N-acetyl-L-cysteine, lipoic acid, tocopherol and its esters (for example, tocopheryl acetate: DL-a-tocopheryl acetate available from Eisai), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof. Further skin care agents are disclosed in US Publication No. 2007/0020220A1, wherein the components/ingredients are incorporated herein by reference in their entirety.

In some embodiments, the topical formulation may further comprise antiaging ingredients selected from the group consisting of ascorbic acid compounds, vitamin B3 compounds, azelaic acid, butyl hydroxyanisole, gallic acid and its derivatives, glycyrrhizinic acid, hydroquinone, kojic acid, arbutin, mulberry extract, and combinations thereof. In some embodiments, the topical composition or final formulation may comprise Ovaliss ((S)-5,6,6a,7-Tetrahydro-1,2,9,10-tetramethoxy-6-methyl-4H-dibenzo[de,g]quinoline, 1,2-Octanediol, D-Glucopyranose, oligomeric, C10-16-alkyl glycosides, water, ethyl alcohol, and glycerin), Whey protein, MPC (Milk protein complex), Sesaflash (Glycerin, Acrylates copolymer, PVP/polycarbamyl polygly col ester, Hydrolyzed Sesame Protein PG-propyl methylsilanediol), Majestem (glycerin, Leontopodium Alpinum Callus Culture Extract and xanthan gum), or Idealift (butylene glycol, sorbitan laurate, hydroxyethylcellulose, and acetyl dipeptide-1 cetyl ester).

In some embodiments, the topical formulation may further comprises sunblock agents selected from the group consisting of para-aminobenzoic acid (PABA), PABA esters (glyceryl PABA, amyldimethyl PABA and octyldimethyl PABA), benzophenones (oxybenzone and sulisobenzone), cinnamates (octylmethoxy cinnamate and cinoxate), salicylates (homomethyl salicylate) anthranilates, TiO₂, avobenzone, bemotrizinol, bisoctrizole, 3-(4-methylbenzylidene)-camphor, cinoxate, diethylamino hydroxybenzoyl hexyl benzoate, dioxybenzone, drometrizole trisiloxane, ecamsule, ethylhexyl triazone, homosalate, menthyl anthranilate, octocrylene, octyl salicylate, iscotrizinol, isopentenyl-4-methoxycinnamate, octyl-dimethyl-p-aminobenzoic acid, octyl-methoxycinnamate, oxybenzone, polysilicone-15, trolamine salicylate, ZnO, and combinations thereof.

In some embodiments, the topical formulation may comprise a sensation modifying agent selected from the group of a cooling agent, a warming agent, a relaxing or soothing agent, a stimulating or refreshing agent, and combinations thereof.

In some embodiments, the cooling agent is selected from the group consisting of menthol; an isomer of menthol, a menthol derivative; 4-Methyl-3-(1-pyrrolidinyl)-2[5H]-furanone; WS-23, Icilin, Icilin Unilever Analog, 5-methyl-4-(1-pyrrolidinyl)-3-[2H]-furanone; 4,5-dimethyl-3-(1-pyrrolidinyl)-2[5H]-furanone; isopulegol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxas-piro[4,5]decane-2-methanol, menthyl succinate and its alkaline earth metal salts, trimethylcyclohexanol, N-ethyl-2-isopropyl-5-methylcycl ohexanecarb-oxamide, Japanese mint (Mentha arvensis) oil, peppermint oil, menthone, menthone glycerol ketal, menthyl lactate, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxy butyrate, N,2,3-trimethyl-2-(1-methylethyl)-butanamide, spearmint oil and combination thereof.

In some embodiments, the warming agent is selected from the group consisting of polyhydric alcohols, capsaicin, capsicum powder, a capsicum tincture, capsicum extract, capsaicin, hamamalis, homocapsaicin, homodihydrocapsaicin, nonanoyl vanillyl amide, nonanoic acid vanillyl ether, vanillyl alcohol alkyl ether derivatives, such as vanillyl ethyl ether, vanillyl butyl ether, vanillyl pentyl ether, and vanillyl hexyl ether, isovanillyl alcohol alkyl ethers, ethylvanillyl alcohol alkyl ethers, veratryl alcohol derivatives, substituted benzyl alcohol derivatives, substituted benzyl alcohol alkyl ethers, vanillin propylene glycol acetal, ethylvanillin propylene glycol acetal, ginger extract, ginger oil, gingeol, gingeron, and combination thereof.

In some embodiments, the relaxing or soothing agent is selected from the group consisting of herb extracts, aloe vera, alpha bisabolol, D-panthenol, allantoin, hamamelis, chamomile, yarrow; calendula, comfrey, witch hazel and other astringents, sea weed, and oat extracts; oils, selected from the group consisting of: almond oil, avocado oil, and comfrey; and essential oils, selected from the group consisting of: cardamone, eucalyptus, Mentha piperita (peppermint), hyssop, and rosemary; waxy or unctuous substances selected from the group consisting of: lanolin or vaseline jelly, minerals, selected from the group consisting of: zinc oxide, calamine and selenium; vitamins, selected from the group consisting of: tocopheryl acetate (vitamin E), and pharmaceutical agents selected from the group consisting of: analgesics, anesthetics, anti-inflammatory agents, and anti-histamines, and muscle relaxants; menthol, camphor, eugenol, eucalyptol, safrol, methyl salicylate, menthyl lactate, menthyl ethoxyacetate, menthone glycerinacetal, 3-1-menthoxypropane-1,2-diol, ethyl 1-menthyl carbonate, (1S,3S,4R)-p-menth-8-en-3-ol, menthyl pyrrolidone carboxylate, N-substituted-p-menthane-3-carboxamides hamamelis extract, ginger oil, and combination thereof.

In some embodiments, the stimulating or refreshing agent is selected from the group consisting of alcohol, L-menthol, camphor, menthe oil, capsicum extract, capsaicin, benzyl nicotinate, salicylate, glycol salicylate, acetyl choline, serotonin, histamine, a prostaglandin, a neurotransmitter, a CNS stimulant, caffeine, quinine, and combination thereof.

In some embodiments, the composition has a shelf life of about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, or any individual value or any range between any two values therein.

The topical compositions may further be configured, adapted, made and/or used in any manner described herein with respect to the method of making and the method of using the topical compositions.

In another aspect of the present subject matter, embodiments disclosed herein are directed to methods of treating a tissue, e.g., with the topical compositions described herein. The method may comprising topically administering, to a tissue of a patient, a composition comprising deconstructed matrisome including one or more enzymatically fragmented peptides (i.e., matrikines), wherein the one or more enzymatically fragmented peptides promote one or more of tissue homeostasis, tissue repair, and tissue regeneration. In some embodiments, the composition is topically applied to an epithelium of the tissue (e.g., a skin surface). In some embodiments, the composition further comprises one or more excipients (e.g., a pharmaceutically acceptable excipient or a cosmetically acceptable excipient). In some embodiments, the deconstructed matrisome is enzymatically fragmented to generate the one or more peptides through proteolysis (i.e., enzymatic degradation) by proteases of one or more deconstructed matrisome components. In some embodiments, the one or more enzymatically fragmented peptides are configured to retain cell signaling ability, thereby promoting one or more of tissue homeostasis, tissue repair, and tissue regeneration. Further, the composition may include any of the components, features, characteristics, and/or variations as described fully herein.

In some embodiments, administering the composition promotes and/or improves at least one characteristic of the tissue.

In some embodiments, the composition increases keratin gene expression. In some embodiments, the composition increases hydration and/or moisturization. In some embodiments, the composition increases tissue regeneration to reduce or prevent aging effects such as laxity, wrinkling, and sagging. In some embodiments, the composition increases tissue regeneration to reduce acne scarring. In some embodiments, the composition promotes pore size reduction and or improve skin tone.

In some embodiments, the composition reduces dermal redness. In some embodiments, the composition promotes scar healing and/or reduces scar formation. In some embodiments, the composition promotes healing and/or recovery from a wound or burn. For example, the burn may be a burn from heat, a chemical burn, and/or photodamage. In some embodiments, the composition reduces skin discoloration. For example, discoloration may be caused by scars, redness, and/or sunspots.

In some embodiments, the composition lowers a pH of a tissue surface to reduce or eliminate pathogen presence or growth. In some embodiments, the composition may lower the pH of the tissue surface to less than about 6.0, less than about 5.5, less than about 5.0, less than about 4.5, less than about 4.0, and/or individual values or ranges therebetween.

In embodiments described herein, improving the look or appearance of skin is an improvement in a characteristic of the skin. In some embodiments, the characteristic of the skin is selected from the group consisting of firmness, elasticity, fine lines, wrinkles, skin texture, skin tone, appearance, and any combination thereof. In some embodiments, improving the look of the skin results in smoother, firmer, young-looking skin. In some embodiments, improving the look of the skin results in a brighter complexion, improved texture, even-looking skin, and/or softer skin. In some embodiments, improving the appearance of the skin results in improvement of discoloration, disappearance of blemishes, and/or decreased redness. In some embodiments, improving the appearance of the skin results in an anti-inflammatory effect.

In some embodiments, treating the tissue such as skin with a topical composition as described herein results in an increase in expression of keratins, wherein the keratins comprise keratin 1, keratin 2, keratin 9, and/or keratin 10. In some embodiments, the topical composition results in about 300% increase in expression of keratins. However, the topical composition may result in an increase in keratin expression of about 50%, about 100%, about 200%, about 300%, about 400%, greater than about 400%, or additional values or ranges therebetween.

In some embodiments, treating the tissue such as skin with a topical composition as described herein results in an increase in cell regeneration, wherein cell regeneration is measured by restoration of the epithelial barrier. In some embodiments, the topical composition results in about 225% increase in cell regeneration. However, the topical composition may result in an increase in cell regeneration of about 50%, about 100%, about 200%, about 300%, about 400%, greater than about 400%, or additional values or ranges therebetween.

In some embodiments, treating the tissue such as skin with a topical composition as described herein results in a decrease in wound diameter. In some embodiments, the topical composition results in about 400% decrease in wound diameter. However, the topical composition may result in a decrease in wound diameter of about 50%, about 100%, about 200%, about 300%, about 400%, about 500%, about 600%, greater than about 600%, or additional values or ranges therebetween.

In some embodiments, treating the tissue such as skin with a topical composition as described herein results in accelerated healing. In some embodiments, the topical composition results in about 7 fold acceleration in healing. However, the topical composition may result in an acceleration in healing of about 2 fold, about 3 fold, about 4 fold, about 5 fold, about 6 fold, about 7 fold, about 8 fold, about 9 fold, about 10 fold, greater than about 10 fold, or additional values or ranges therebetween.

In some embodiments, treating the tissue such as skin with a topical composition as described herein results in a decrease in skin redness. In some embodiments, the topical composition results in about 80% decrease skin redness. However, the topical composition may result in a decrease in skin redness of about 20%, about 40%, about 60%, about 80%, about 100%, about 200%, about 300%, greater than about 300%, or additional values or ranges therebetween.

In some embodiments, treating the tissue such as skin with a topical composition as described herein results in an increase in scar healing. In some embodiments, the topical composition results in about 700% decrease skin redness. However, the topical composition may result in a decrease in skin redness of about 100%, about 200%, about 300%, about 400%, about 500%, about 600%, about 700%, about 800%, about 900%, about 1000% greater than about 1000%, or additional values or ranges therebetween.

In some embodiments, the composition may be configured to treat a specific tissue condition or disease in a subject in need thereof. Accordingly, the method may comprise topically administering, to a tissue of the subject, a topical composition as described herein. In some embodiments, treating the tissue such as skin with the topical composition results in an improvement in at least one symptom or characteristic of the tissue condition or disease.

In some embodiments, the tissue condition is selected from acne, eczema, and psoriasis. In some embodiments, the tissue condition is a fibrotic skin condition selected from scleroderma, nephrogenic fibrosing dermopathy, mixed connective tissue disease, scleromyxedema, scleroderma, eosinophilic fasciitis, and combinations thereof.

While the compositions and methods herein are generally described with respect to skin tissue, it should be understood that the composition may be configured for application to another tissue and/or by another route of administration. In each case, the composition may be administered in the conventional manners accordingly.

In some embodiments the composition is a lip balm used to promote repair and regeneration of lips or other mucosal surfaces and may be administered topically to the lips.

In some embodiments the composition is an ophthalmic solution used to minimize or reduce corneal scarring and may be administered to the eyes.

In some embodiments the composition is an edible dietary supplement used to treat gastrointestinal health and regeneration and may be administered orally.

In some embodiments the matrikine composition is a cosmetic product such as a hair styling product, an anti-frizz product, an oil, a nail product, a beautification product, and the like.

In some embodiments the matrikine composition is a personal lubricant.

In some embodiments the composition is a personal care product such as mouth wash, sunscreen, or an after sun care product.

In some embodiments the composition is a hair care product used to promote hair or fur health and may be administered topically to the hair and/or fur. In some embodiments the composition is a hair care product used to promote growth of scalp hair, eyebrows, and eyelashes and may be administered to the scalp, eyebrows, and/or eyelashes. In embodiments described herein, the treatment of the hair results in improvement in a characteristic of the hair. In embodiments described herein, the characteristic of the hair is selected from the group consisting of shine, texture, fullness, smoothness, density, and combinations thereof. In embodiments described herein, improving the appearance of the hair results in smoother hair, softer hair, brighter hair, improved texture of the hair, shiner hair, fuller hair, or more vibrant hair.

In embodiments described herein, the subject is an infant, a child, an adolescent, or an adult. In embodiments described herein, the subject is an veterinary animal.

In some embodiments, the topical compositions and formulations can be applied to the skin one, two, three, four, five or more times each day, and application can be carried out for a period of at least 1 month, 2 months, 3 months, 4 months, 6 months, 8 months or 12 months.

In some embodiments, the topical compositions and formulations may be administered once, as needed, once daily, twice daily, three times a day, once a week, twice a week, every other week, every other day, or the like for one or more dosing cycles. A dosing cycle may comprise administration for about 1 week, about 2 weeks, about 3 weeks, about 4 weeks, about 5 weeks, about 6 weeks, about 7 weeks, about 8 weeks, about 9 weeks, or about 10 weeks. After this cycle, a subsequent cycle may begin approximately 1, 2, 3, 4, 5, 6, 7, 8, 9, 10, 11, or 12 weeks later. The treatment regime may comprise 1, 2, 3, 4, 5, or 6 cycles, each cycle being spaced apart by approximately 1, 2, 3, 4, 5, 6, 7, 8, 9, 10, 11, or 12 weeks.

In some embodiments, the methods may comprise a variety of additional steps including, for example, cleaning the surface tissue at the site of applying, abrading, microdermabrasion, toning, and the like.

The method of treating a tissue further be adapted in any manner described herein with respect to the composition and the method of making the composition.

In another aspect of the present subject matter, embodiments disclosed herein are directed to methods of making the topical compositions described herein. A wide variety of methods may be used for preparing the compositions and formulations described herein. Broadly speaking, the compositions may be prepared by combining the components of the formulation, as described herein, at a temperature and for a time sufficient to provide a pharmaceutically or cosmetically acceptable composition.

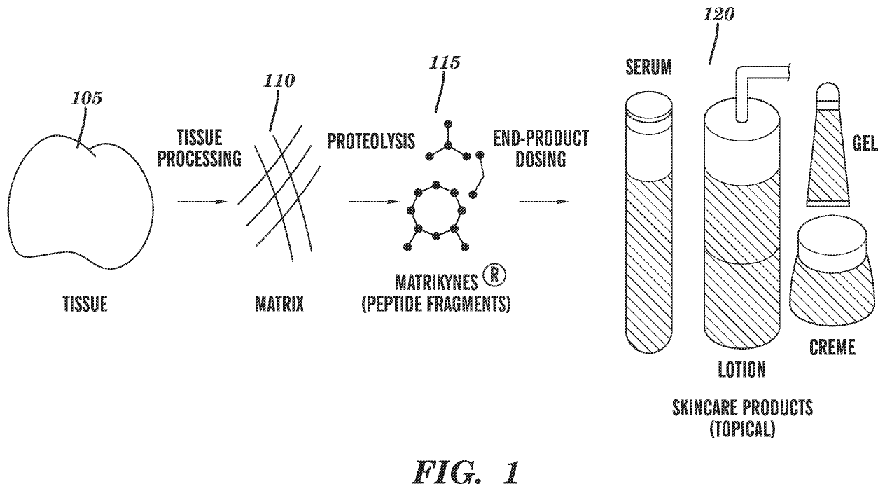

FIG. 1 depicts a diagram of an illustrative method of making the topical composition in accordance with an embodiment. The method may comprises providing 105 a tissue, isolating 110 a decellularized, acellular tissue-specific extracellular matrix from biological tissue, fragmenting and solubilizing 115 the tissue-specific extracellular matrix using one or more enzymes to produce a deconstructed matrisome solution including one or more enzymatically fragmented peptides (i.e., matrikines), and combining 120 the matrix solution with one or more excipients (e.g., a pharmaceutically acceptable excipient or a cosmetically acceptable excipient) to form the topical composition. In some embodiments, the one or more peptides promote one or more of tissue homeostasis, tissue repair, and tissue regeneration. In some embodiments, the composition is configured for topical application to an epithelium of the tissue (e.g., a skin surface). In some embodiments, the deconstructed matrisome is fragmented to generate the one or more peptides through proteolysis (i.e., enzymatic degradation) of one or more deconstructed matrisome components. Further, the composition may include any of the components, features, characteristics, and/or variations as described fully herein.

According to an exemplary embodiment, tissue is procured and immediately frozen and prepared for sectioning. Frozen blocks are then sectioned longitudinally into thin (200 μm-1 mm) slices showing the entire cross-section of the tissue. Portions of the tissue may be dissected and separated from the thin slices prior to decellularization. The tissues are treated using a sequence of chemical, detergent, and enzymatic washes. Each wash is followed by de-ionized water washes. In some embodiments, each region is decellularized by serial washes up to 12 hours followed by enzymatic digestions.

The described process may be modified or adapted for various tissues described herein. Tissue sections are decellularized by the introduction of one or more of deionized water, hypertonic salines, enzymes, detergents, and acids. In an exemplary embodiment, tissue sections are decellularized by using a sequence of chemical, detergent, and enzymatic washes. Each wash may be followed by de-ionized water washes.

Following decellularization, the decellularized material is snap frozen in liquid nitrogen, pulverized, milled, and lyophilized to obtain a fine ECM powder. In some embodiments, the ECM powder is digested using an enzymatic agent. In some embodiments an ECM solution is produced from the ECM powder. The resulting digest may neutralized, frozen, and thawed to obtain ECM solution. In some embodiments, the solubilizing step may not be performed and the ECM material may be utilized in its powder form.

The ECM solution or ECM powder may be combined with an excipient such as a pharmaceutically acceptable excipient and/or a cosmetically acceptable excipient to produce the topical composition. The ECM material and the excipient may be combined, mixed, and/or homogenized. Further, any number of additional materials, such as the agents and/or inactive ingredients described herein with respect to the topical formulation.

In some embodiments, the ECM solution may be reconstituted into another form before being combined with additional materials. The ECM material may be reconstituted into a hydrogel or another format by adding a reagent such as a buffer to adjust the ionic strength and the pH of the solution. In some embodiments, the reagent comprises one or more of a neutral buffer, a basic buffer, a base, and an acid. For example, a neutral buffer may comprise Phosphate Buffered Saline (PBS), TAPSO (3-[N-tris(hydroxymethyl)methylamino]-2-hydroxypropanesulfonic acid), HEPES (4-(2-hydroxyethyl)-1-piperazineethanesulfoni c acid), TES (2-[[1,3-dihydroxy-2-(hydroxymethyl)propan-2-yl]amino]ethanesulfonic acid), and/or MOPS (3-(N-morpholino)propanesulfonic acid). For example, a basic buffer may comprise carbonate bicarbonate, TAPS ([tris(hydroxymethyl)methylamino]propanesulfonic acid), Bicine (2-(bis(2-hydroxyethyl)amino)acetic acid), Tris (tris(hydroxymethyl)aminomethane), and/or Tricine (N-[tris(hydroxymethyl))methyl]glycine). For example, a base may comprise Sodium Hydroxide (NaOH). For example, an acid may comprise Hydrochloric Acid (HCl) or Acetic Acid. In additional embodiments, the reagent may comprise deionized water. However, additional or alternative reagents may be provided to convert the ECM material into various forms, as would be known to a person having an ordinary level of skill in the art. In still additional embodiments, a reagent is not required.

In some embodiments, the process may be further adapted based on the properties of the tissue. In some embodiments, a higher content of connective tissue and/or the greater mechanical stiffness may require longer digestion than would be required for other tissues. In some embodiments, to form a solution as described, the ECM powder is digested with an enzymatic agent for about 1 hour, about 2 hours, about 3 hours, about 4 hours, about 5 hours, greater than about 5 hours, or individual values or ranges therebetween.

In some embodiments, a plurality of deconstructed matrisome solutions may be formed by the manner described herein and used to form the topical composition. In some embodiments, the topical composition may comprise one, two, three, four, five, or more different deconstructed matrisome solutions.

In some embodiments, the topical composition has a shelf life of about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, greater than about 10 years, or any individual value or any range between any two values therein.

The topical composition may be characterized by any of the components, concentrations thereof, and/or changes thereof from normal as summarized in Table 1, Table 2, and Table 3, Table 4, Table 5 and Table 6. However, these compositions are exemplary in nature and the ECM profiles may vary therefrom as to any number of components. For example, the composition of the substrate may vary from the described concentration values and/or ranges by about 10%, about 20%, about 30%, greater than 30%, or individual values or ranges therebetween.

As described herein, the matrikines in the topical composition may be configured to support tissue regeneration and healing. Further, the matrikines may be configured to facilitate growth and proliferation of the human skin fibroblasts in a manner consistent with tissue healing. Accordingly, the matrikines may induce gene expression, growth factor secretion, and other characteristics in a manner consistent with tissue healing. However, the matrikines may be configured to support a variety of additional cell types found in the skin, i.e., native cells.

The method of making a ECM substrate may further be adapted in any manner described herein with respect to the ECM substrate and the method of using the ECM substrate.

The subject matter is now described with reference to the following examples. These examples are provided for the purpose of illustration only and the claims should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### Examples

The disclosures of each and every patent, patent application, publication, and accession number cited herein are hereby incorporated herein by reference in their entirety.

While present disclosure has been disclosed with reference to various embodiments, it is apparent that other embodiments and variations of these may be devised by others skilled in the art without departing from the true spirit and scope of the disclosure. The appended claims are intended to be construed to include all such embodiments and equivalent variations.

### Example 1: Gene Expression of Normal Human Dermal Fibroblast (NHDF) Cultured in Different Deconstructed Matrisome Components

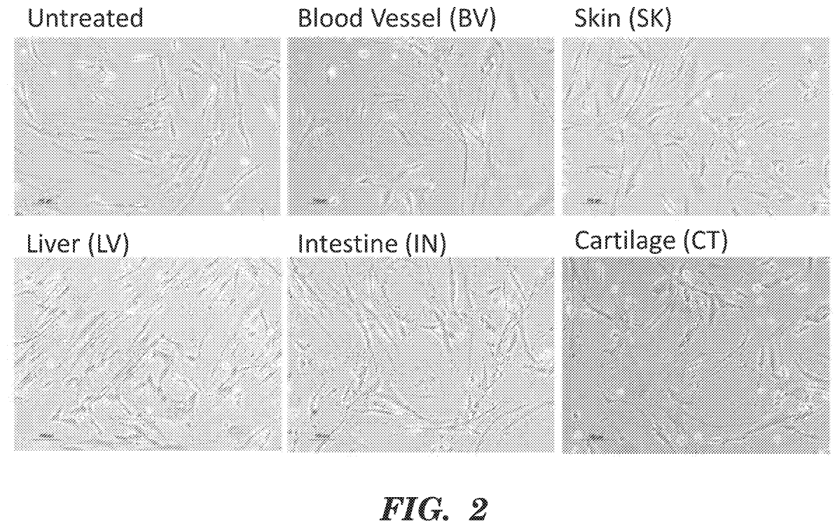

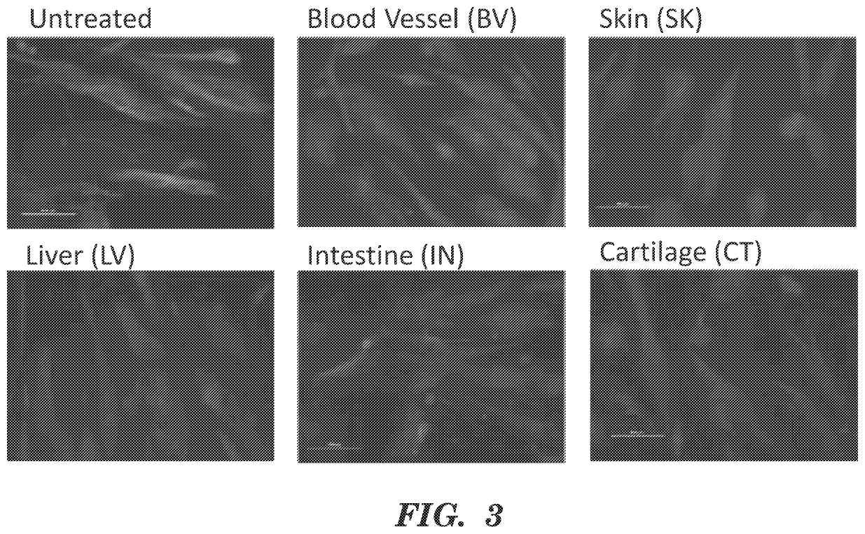

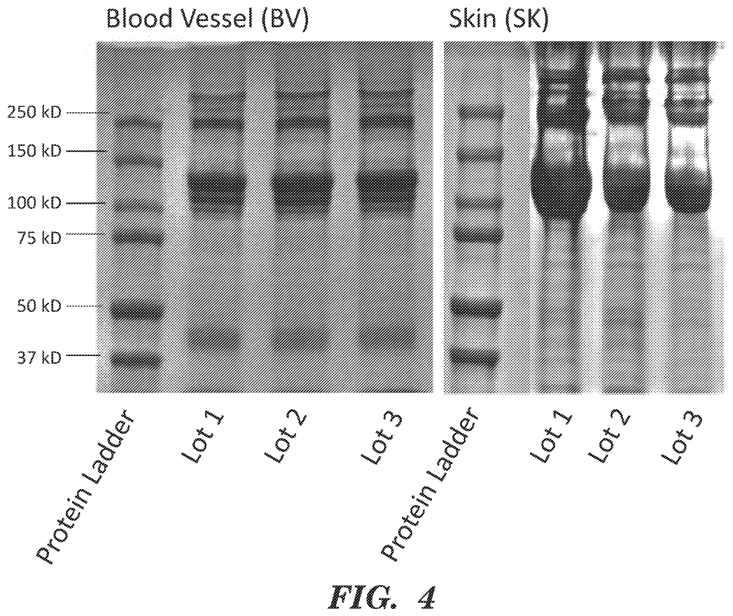

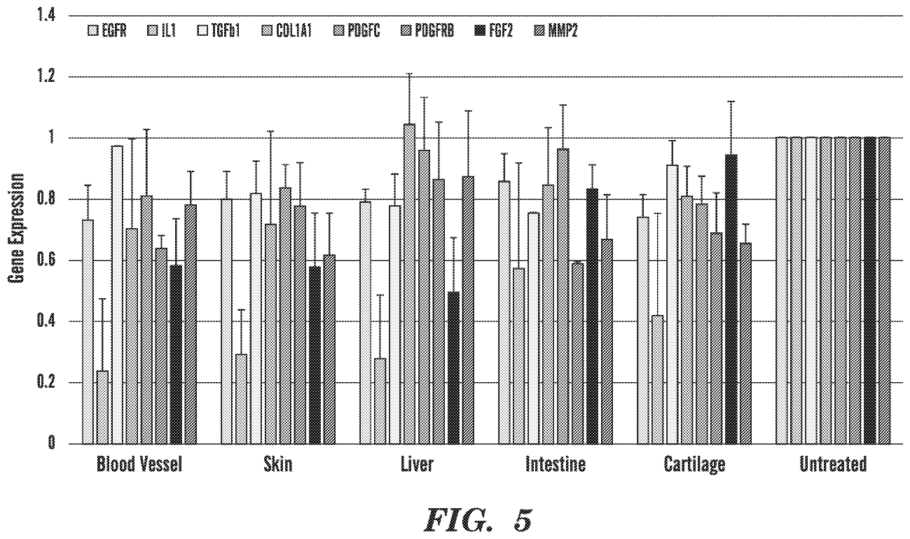

NHDFs were cultured on plastic as well as in blood vessel-, skin-, liver-, intestine-, and cartilage-specific extracellular matrix components, as seen by light microscope (FIG. 2) and immunofluorescent microscopy (FIG. 3; SDS-PAGE of blood vessel, BV, and skin, SK, extracellular matrix shown in FIG. 4). Following RNA extraction and qPCR, the presence of genes related to wound-healing and scarring (including EGFR, IL1, TGFb1, COLA1, PDGFC, PDGFRB, FGF2, MMP2) were quantified. Data from blood vessel and skin were averaged across three lots for both of these tissue types. NHDFs showed decreased expression of wound healing genes across all tissue-specific component conditions in comparison to plastic (no deconstructed matrisome components) (FIG. 5). Interleukin-1 gene expression was reduced in the presence of deconstructed matrisome components suggesting that these components may inhibit inflammatory responses. Genes may be induced or expressed differently in each specific tissue type.

### Example 2: Characterization of Matrikine Formula 1

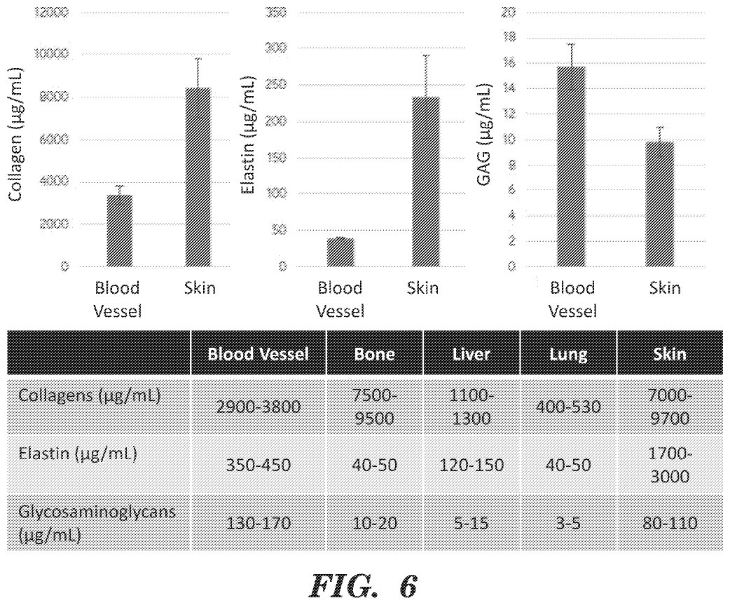

Biochemical analysis of blood vessel and skin extracellular matrix components, or matrikines. Extracellular matrix components from blood vessel and skin were analyzed for protein expression. Minimal variability across lots within each tissue type was observed (FIG. 6).

### Example 3: Matrikynes Increase Skin Healing

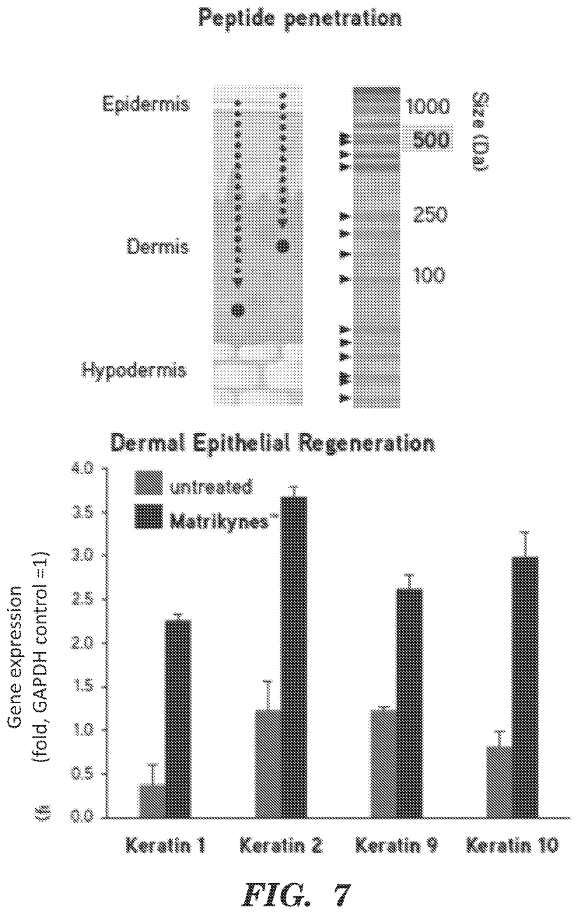

Interactions between the matrikines and the microbiome are instrumental to healthy barrier function and protection from pathogens. The intrinsic bioactivity of matrikines regulate immunologic response, instruct dermal epithelial cell organization and restore normal skin tissue architecture. Matrikynes® lower skin surface pH (5.5) to make skin inhospitable to pathogens and produce antimicrobial peptides that limit growth of pathogenic microbes on skin. Matrikynes® containing extracellular matrix fragments smaller than 500 Da allow for penetration into the dermis (FIG. 7). The experiments and measurements here quantify this regeneration.

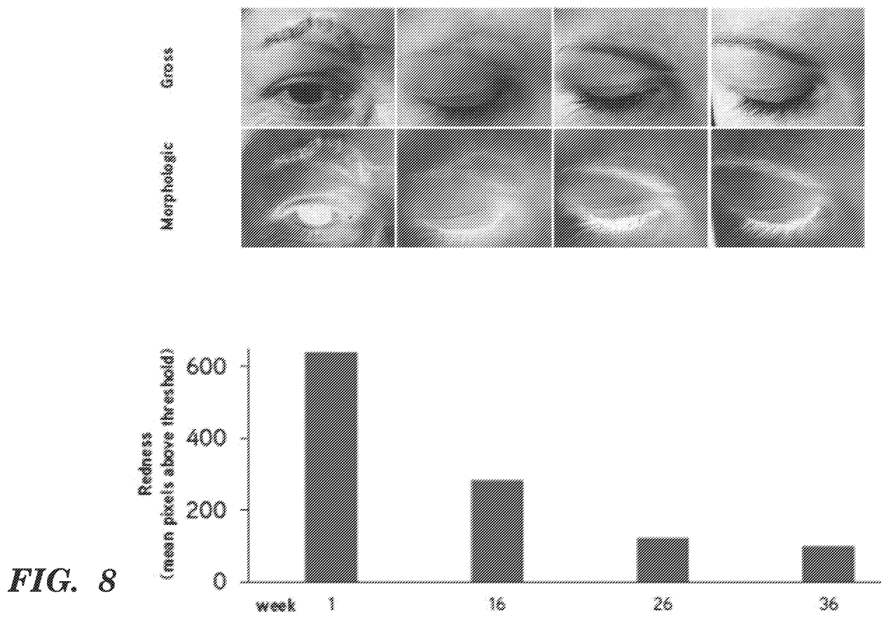

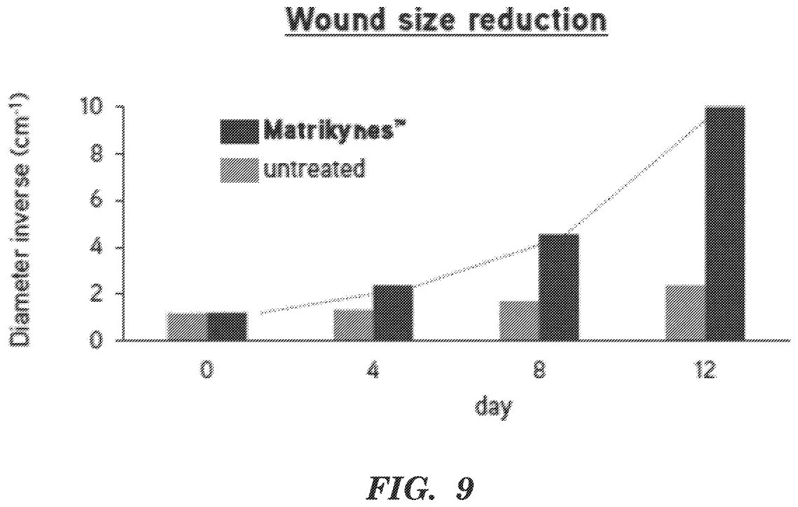

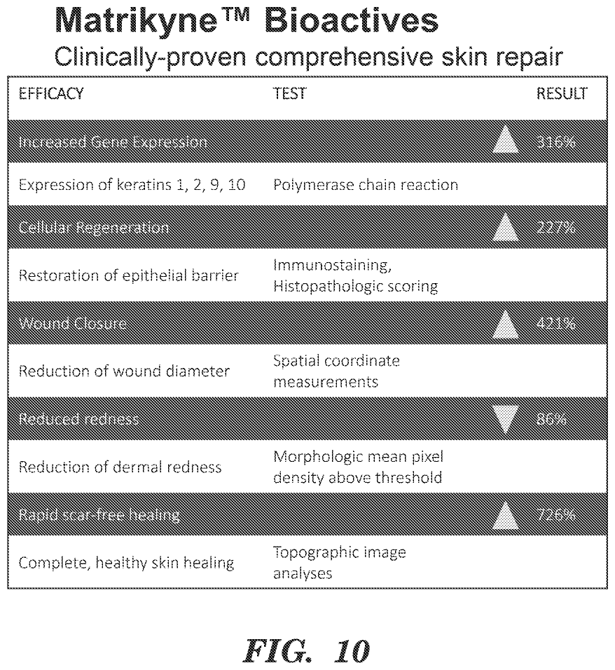

Matrikynes®, according to Formula 1 (Table 1), promote epithelial regeneration, which is measured by an increase in keratin expression (FIG. 7). Matrikynes® improve skin repair and healing and reduce the appearance of scars. Application of Matrikynes® showed about an 86% reduction in redness as measured by mean pixels above threshold over thirty six weeks (FIG. 8). Matrikynes® reduce the size of wounds as compared to untreated tissue by 421% with 7.3 times faster healing (FIG. 9). In summary, treatment with Matrikynes® leads to a 316% increase in keratin expression, a 227% increase in cellular regeneration compared to untreated tissue, a 421% increase in wound closure, an 86% decrease in wound redness, and a 726% increase in skin healing (FIG. 10).

### Example 4: Method of Making Matrikynes®

Tissue-specific decellularized extracellular matrix can be formulated into skin care formulations for topical application. The first step in creating any of these products is to isolate tissue and collect tissue samples. The tissue from these samples then undergoes a cell removal process, or decellularization, and the extracellular matrix is them isolated away from the rest of the cellular components. Once the extracellular matrix is isolated it is then fragmented by enzymatic degradation using a number of proteases and processed into a powder for reconstitution in a topical formulation for skin treatment.

### Example 5: Matrikynes Modulate Cellular Activity

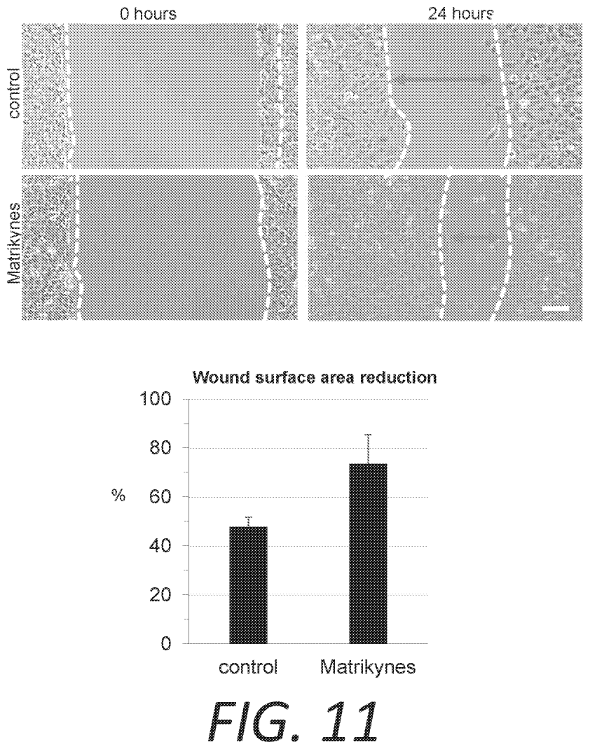

Primary human epithelial cells were cultured for 4 days (FIG. 11). Scratch assay was performed with Matrikynes® (Formula 1, Table 1), and without Matrikynes® (control) to evaluate effects of Matrikynes®, which after 24 hours increased cellular migration, proliferation, and wound closure compared to untreated controls (scale bar: 100 μm).

### Example 6: Human Repeat Insult Patch Test (HRIPT)

Substances that come in contact with human skin must be evaluated for propensity to irritate and/or sensitize. A reproducible, standardized, quantitative patch evaluation procedure must be used to demonstrate that a particular material can be applied safely to human skin without significant risk of adverse reactions.

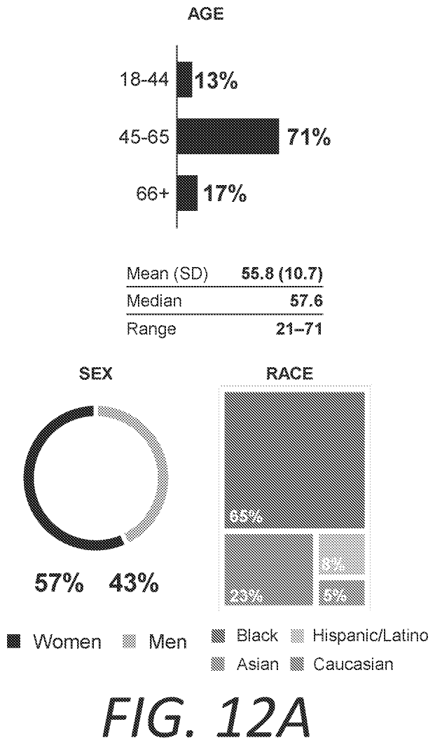

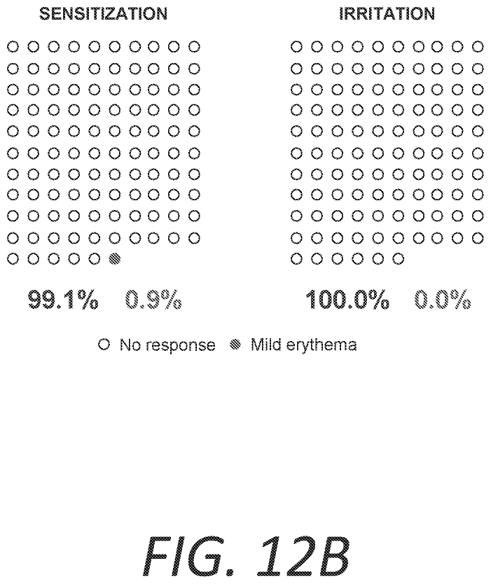

Sensitization was determined by repeated topical applications to the skin of human subjects under controlled patch test conditions. Repeated insult patch evaluation is a predictive patch study that can detect irritant reactions and weak sensitizers that require multiple applications to induce a cell-mediated (type IV) immune response sufficient to cause an allergic reaction. A reproducible, standardized, quantitative patch evaluation procedure was used to demonstrate that Matrikynes® at 2.5% by weight can be applied safely to human skin without adverse reaction (FIGS. 12A and 12B; N=106 human subjects). Matrikynes® of Formula I (Table 1) were used in the HRIPT.

### Example 7: Case Studies in Wound and Scar Healing

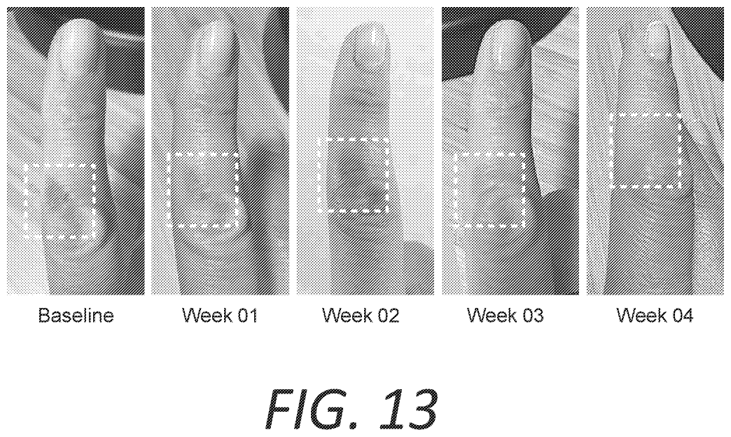

Superficial Burn: Methods: Base cream containing Matrikynes® (0.1% by weight; Formula 1, Table 1) was applied twice daily for four (4) weeks to the site of a superficial burn on the index finger. No other medications or products were used. Results: Gross photography showed reduced redness, skin repair, and scar-free healing after four (4) weeks (FIG. 13).

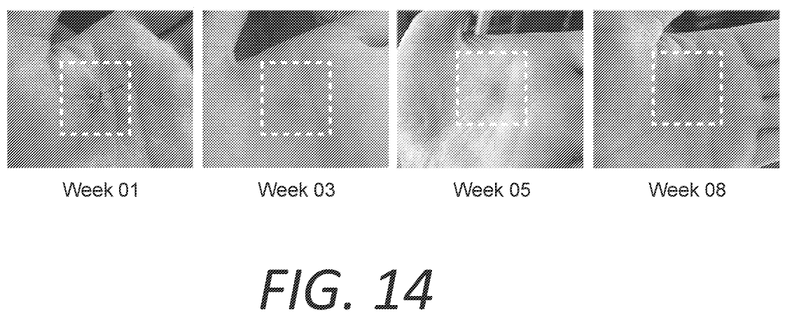

Wound Healing: Methods: Base cream containing Matrikynes® (0.1% by weight; Formula 1, Table 1) was applied twice daily for eight (8) weeks to the site of a wound closed with surgical suture on the palm of the hand. No other medications or products were used. Results: Gross photography showed reduced redness and healing after eight (8) weeks (FIG. 14).

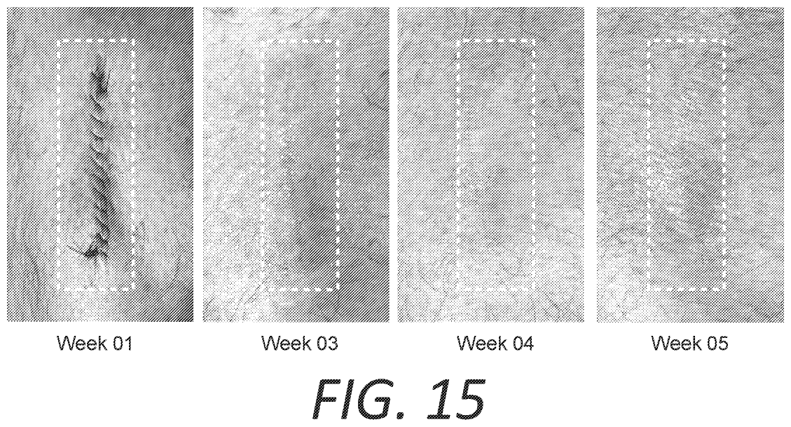

Wound Healing with Surgical Suture: Methods: Base cream containing Matrikynes® (0.1% by weight; Formula 1, Table 1) was applied twice daily over the course of five (5) weeks to the site of a wound closed with surgical suture on the lower leg. No other medications or products were used. Results: Gross photography showed reduced redness and healing without scar after five (5) weeks (FIG. 15).

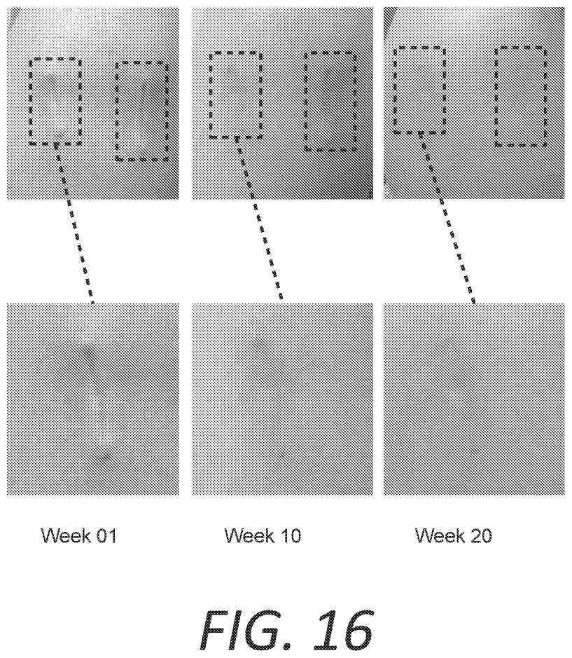

Scar Reduction: Methods: Base cream containing Matrikynes® (0.1% by weight; Formula 1, Table 1) was applied twice daily for twenty (20) weeks to the site of post-surgical fine line scars on the upper arm. No other medications or products were used. Results: Gross photography showed reduced raise of scars, smoother skin texture at the scar sites, and improved appearance after twenty (20) weeks (FIG. 16).

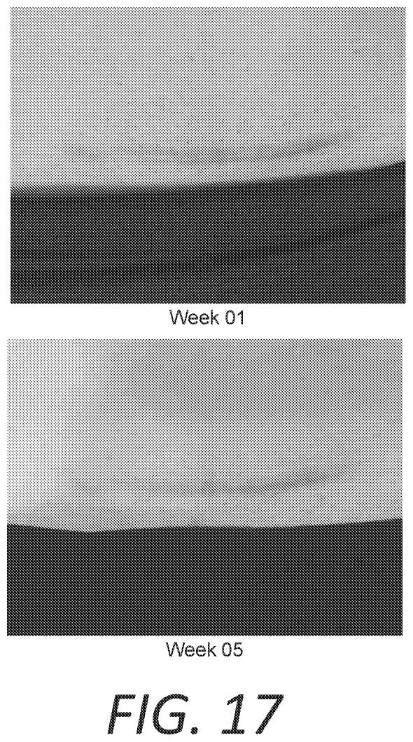

Scar Reduction: Methods: Base cream containing Matrikynes® (0.1% by weight; Formula 1, Table 1) was applied twice daily for five (5) weeks to a Caesarian section scar. No other medications or products were used. Results: Gross photography showed reduced raise and length of scar, reduced discoloration, and improved appearance after five (5) weeks (FIG. 17).

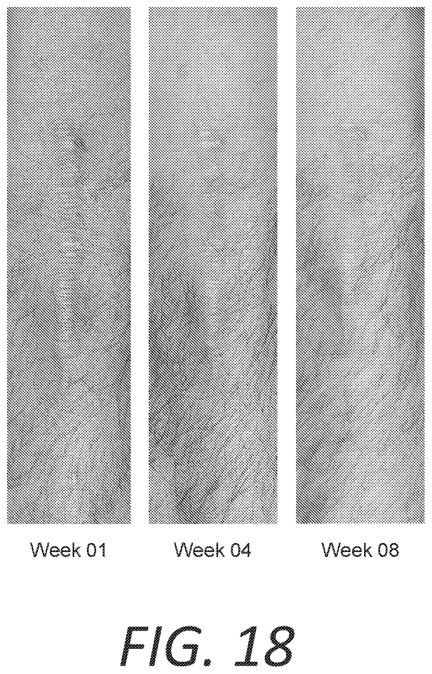

Scar Reduction: Methods: Base cream containing Matrikynes® (0.1% by weight; Formula 1, Table 1) was applied twice daily for eight (8) weeks to a seven-year-old ACL knee surgery scar. No other medications or products were used. Results: Gross photography showed reduced raise and length of scar, reduced wrinkles, and improved appearance after eight (8) weeks (FIG. 18).

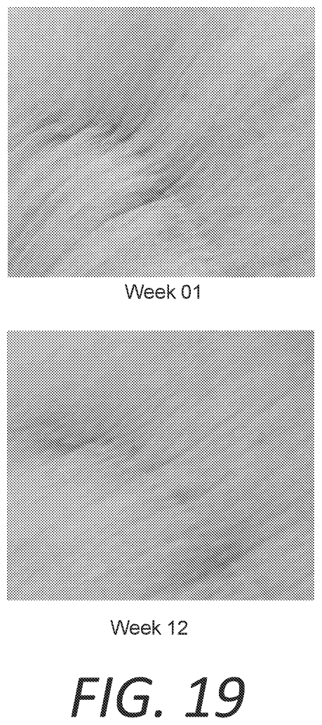

Scar Reduction: Methods: Base cream containing Matrikynes® (0.1% by weight; Formula 1, Table 1) was applied twice daily for twelve (12) weeks to a two-year-old hip replacement surgery scar in a 61 year old female subject. No other medications or products were used. Results: Gross photography showed visible diminished appearance of scar after twelve (12) weeks (FIG. 19).

### Example 8: Case Studies in Treating Skin Conditions

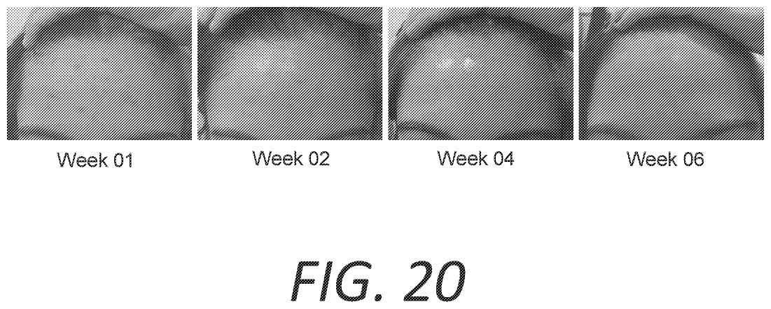

Acne vulgaris study 1: Methods: Base cream containing Matrikynes® (0.1% by weight; Formula 1, Table 1) was applied twice daily for six (6) weeks to areas of the face with acne lesions. No other medications or products were used. Results: Gross photography showed resolution of acne lesions, reduced post-inflammatory erythema, and improved skin tone after six (6) weeks (FIG. 20).

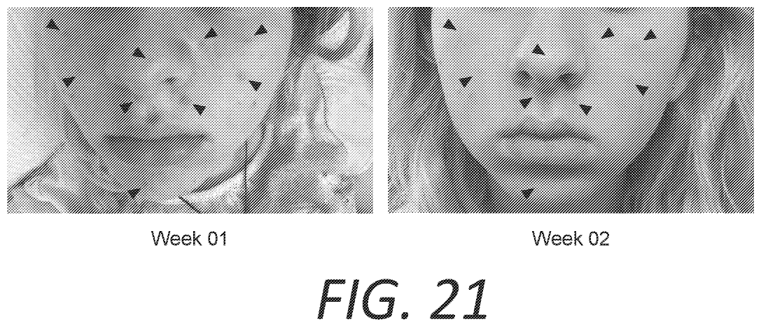

Acne vulgaris study 2: Methods: Base cream containing Matrikynes® (0.1% by weight; Formula 1, Table 1) was applied twice daily for two (2) weeks to areas of the face with acne lesions. No other medications or products were used. Results: Gross photography showed resolution of acne lesions, reduced post-inflammatory erythema, and improved skin tone and texture after two (2) weeks (FIG. 21).

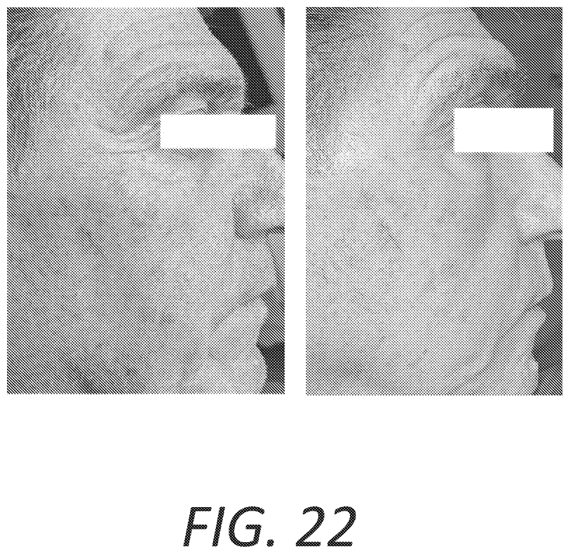

Anti-aging and anti-wrinkle: Methods: Base cream containing Matrikynes® (0.1% by weight; Formula 1, Table 1) was applied twice daily for six (6) weeks to the face. No other medications or products were used. Results: Gross photography showed reduced appearance of: photodamage, redness, and fine lines after two (2) weeks (FIG. 22).

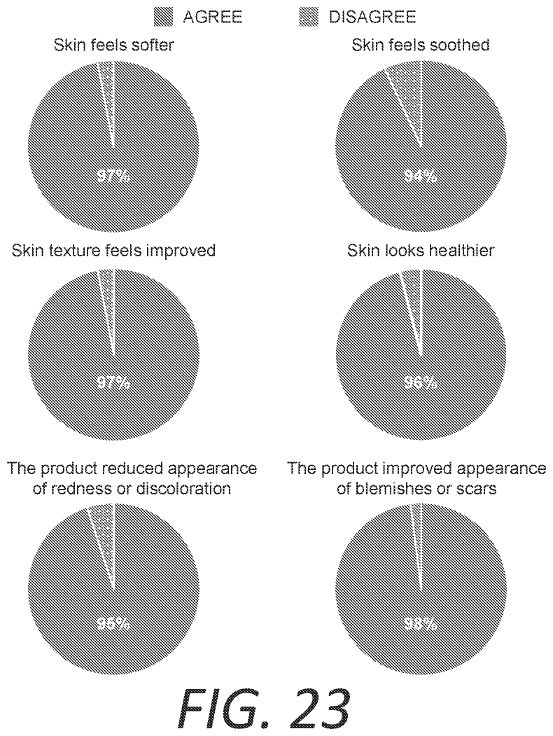

Subjects using Matrikynes® (0.1% by weight; Formula 1, Table 1) for treatment of skin conditions reported high levels of satisfaction (FIG. 23). Subjects found that skin felt softer, skin felt smoothed, skin texture felt improved, and skin looked healthier. Subjects agreed that the product reduced appearance of redness or discoloration and improved appearance of blemishes and scars (FIG. 23).

### Example 9: Anti Aging Properties of Matrikynes®

Objective and Methods: Subjects aged 35 to 65 years of age, will be observed for skin changes after treatment with the Matrikyne® product. The clinical study will evaluate the parameters listed below. A baseline for each of the parameters measured will be established after a 5 day washout period. Subjects will then be given the Matrikyne® product to use according to instructions for the duration of the study. Subjects will return to the clinic after 1 hour, 4 hours, 8 hours, 4 weeks and 8 weeks for evaluation.

Parameters: 1) reduction in the appearance of fine lines and wrinkles (globally), measured by VAESTRO analysis of photos obtained with Canfield VISIA CR; 2) subjective, measured by a questionnaire; 3) improvement in skin firmness and elasticity, measured by cutometer; 4) improvement in skin hydration/moisturization, measured by corneometer; 5) improvement in skin barrier function, measured by TEWL; 6) improvement in the appearance of hyperpigmentation/age spots, measured by VAESTRO analysis of photos with Canfield VISIA CR; 7) improvement in skin density, measured by ultrasound; 8) reduction in the appearance of fine lines and wrinkles in the Crow's feet, measured by primos 3D; 9) repair of skin barrier function, measured by TWEL with tape stripping.

Anticipated Results: Subjects treated with Matrikyne® will demonstrate a reduction in the appearance of fine lines and wrinkles (globally as well as Crow's feet), will subjectively report an improvement in skin condition, an improvement in skin firmness and elasticity, an improvement in skin hydration/moisturization, an improvement in skin barrier function, an improvement in the appearance of hyperpigmentation/age spots, an improvement in skin density, and repair of skin barrier function.

What is claimed is:

1. A composition for topical administration to an epithelium, the composition comprising:
a deconstructed matrisome including one or more enzymatically fragmented peptides derived from at least one biological tissue; and
one or more pharmaceutically acceptable or cosmetically acceptable excipients,
wherein the deconstructed matrisome is in an amount from about 0.1% by weight to about 15% by weight of the composition; and
wherein the one or more enzymatically fragmented peptides are configured to retain cell signaling ability, thereby promoting one or more of tissue homeostasis, tissue repair, and tissue regeneration.

2. The composition of claim 1, wherein the deconstructed matrisome is in an amount from about 0.1% by weight to about 2.5% by weight of the composition.

3. The composition of claim 1, wherein the enzymatically fragmented peptides are sized and configured to be absorbed through one or more dermal layers.

4. The composition of claim 3, wherein the one or more dermal layers comprise epidermis and dermis.

5. The composition of claim 1, where in the enzymatically fragmented peptides have a size of less than about 500 Da.

6. The composition of claim 6, where in the enzymatically fragmented peptides have a size of less than about 250 Da.

7. The composition of claim 1, wherein the composition has a pH of less than about 6.0.

8. The composition of claim 1, wherein the deconstructed matrisome comprises one or more of a solution and a powder.

9. The composition of claim 1, wherein the deconstructed matrisome comprises one or more fragments of collagens, glycoproteins, proteoglycans, glycosaminoglycans, laminins, extracellular matrix associated proteins, soluble growth factors, inflammatory cytokines and chemokines, and immune mediators.

10. The composition of claim 9, wherein the fragments of collagens are in an amount from about 400 ng/mL to about 9700 ng/mL.

11. The composition of claim 9, wherein the fragments of collagens comprise collagen type IV in an amount from about 2 ng/mL to about 24 ng/mL.

12. The composition of claim 9, wherein the fragments of glycosaminoglycans are in an amount from about 3 μg/mL to about 170 ng/mL.

13. The composition of claim 1, wherein the deconstructed matrisome comprises one or more fragments of collagens, glycoproteins, proteoglycans, elastins, matrisome secreted factors, structural proteins, growth factors, and ECM regulators.

14. The composition of claim 13, wherein the fragments of collagens are in an amount from about 400 ng/mL to about 9700 ng/mL.

15. The composition of claim 13, wherein the fragments of elastins are in an amount of about 40 ng/mL to about 3000 ng/mL.

16. The composition of claim 13,
wherein the one or more fragments of collagens comprise collagen type I alpha 1 chain, collagen type III alpha 1 chain, and collagen type V alpha 2 chain;
wherein one or more fragments of glycoproteins comprise fibrillar collagen NC1 domain-containing protein, fibrillin 1, and microfibril associated protein 4;
wherein the one or more fragments of proteoglycans comprise heparan sulfate proteoglycan 2;
wherein the one or more fragments of elastins comprise elastin isoform;
wherein the one or more fragments of structural proteins comprise actin gamma 2 and filamin A; and
wherein the one or more fragments of growth factors comprise latent transforming growth factor beta binding protein 4.

17. The composition of claim 13,
wherein the one or more fragments of collagens comprise collagen type I alpha 1 chain, collagen type I alpha 2 chain, collagen type II alpha 1 chain, collagen type III alpha 1 chain, collagen type V alpha 1 chain, collagen type V alpha 2 chain, collagen type VI alpha 2 chain, collagen type VI alpha 3 chain, collagen type VIII alpha 1 chain, collagen type IX alpha 2 chain, collagen type XI alpha 1 chain, collagen type XI alpha 2 chain, collagen type XII alpha 2 chain, and collagen type XIV alpha 1 chain;
wherein the one or more fragments of glycoproteins comprise fibrillin 1, adipocyte enhancer binding protein 1, alpha-2-Heremans-Schmid glycoprotein, biglycan, extracellular matrix protein 2, fibrinogen beta chain, fibrinogen gamma chain, fibronectin 1, osteonectin, periostin, tenascin C, tenascin N, thrombospondin 1, transforming growth factor beta induced, and vitronectin;
wherein the one or more fragments of proteoglycans comprise heparan sulfate proteoglycan 2, aggrecan core protein, asporin, decorin, fibromodulin, lumican, mimecan, osteoglycan, osteomodulin, and proline/arginine-rich end leucine-rich repeat protein;
wherein the one or more fragments of elastins comprise elastin;
wherein the one or more fragments of matrisome secreted factors comprise albumin, annexin A2, chitinase, collectin subfamily member 12, creatine kinase B, olfactomedin;
wherein the one or more fragments of ECM regulators are coagulation factor IX, coagulation factor X, inter-alpha (globulin) inhibitor H4, prothrombin, and serpin peptidase inhibitor Glade F; and
wherein the one or more fragments of structural proteins are actin gamma 2 and vimentin.

18. The composition of claim 13,
wherein the one or more fragments of collagens comprise collagen type I alpha 1 chain, collagen type I alpha 2 chain, collagen type II alpha 1 chain, collagen type III alpha 1 chain, collagen type IV alpha 1 chain, collagen type IV alpha 2 chain, collagen type V alpha 1 chain, collagen type V alpha 2 chain, collagen type VI alpha 2 chain, collagen type VI alpha 3 chain, collagen type VI alpha 5 chain, collagen type VIII alpha 1 chain, and collagen type VIII alpha 2 chain;
wherein the one or more fragments of glycoproteins comprise dermatopontin, fibrillin 1, microfibril-associate protein 4, and periostin;
wherein the one or more fragments of proteoglycans comprise asporin and heparan sulfate proteoglycan 2;
wherein the one or more fragments of elastins comprise elastin isoform;
wherein the one or more fragments of matrisome secreted factors comprise chitinase, collectin subfamily member, trefoil factor 1, and vasoactive intestinal peptide;
wherein the one or more fragments of ECM regulators comprise hyaluronan binding protein 2;
wherein the one or more fragments of structural proteins comprise actin gamma 2 and myosin 11; and
wherein the one or more fragments of growth factors comprise amphiregulin, basic fibroblast growth factor, bone morphogenic protein 4, bone morphogenic protein 7, epidermal growth factor, growth differentiation factor 15, hepatocyte growth factor, insulin-like growth factor binding protein 3, and osteoprotegerin.

19. The composition of claim 13,
wherein the one or more fragments of collagens comprise collagen type I alpha 1 chain, collagen type I alpha 2 chain, collagen type II alpha 1 chain, collagen type III alpha 1 chain, collagen type IV alpha 1 chain, collagen type V alpha 2 chain, collagen type VI alpha 3 chain, collagen type VI alpha 5 chain;
wherein the one or more fragments of glycoproteins comprise fibrillin 1, fibrillin 2, EGF-containing fibulin-like extracellular matrix protein, laminin subunit gamma 1, prostate stem cell antigen, saposin-B-Val, and von Willebrand factor;
wherein the one or more fragments of proteoglycans comprise heparan sulfate proteoglycan;
wherein the one or more fragments of elastins comprise elastin isoform;
wherein the one or more fragments of matrisome secreted factors comprise chitinase, mucin SAC, mucin 6, serum albumin, and trefoil factor 2;
wherein the one or more fragments of ECM regulators comprise granulin precursor;
wherein the one or more fragments of structural proteins comprise actin, keratin 1, keratin 2, keratin 9, keratin 10, myosin heavy chain 9, and tubulin beta chain; and
wherein the one or more fragments of growth factors comprise bone morphogenic protein 4, fibroblast growth factor 2, insulin-like growth factor binding protein 4, macrophage colony-stimulating factor 1 receptor (CD115), and pro-epidermal growth factor.

20. The composition of claim 13,
wherein the one or more fragments of collagens comprise collagen type I alpha 1 chain, collagen type I alpha 2 chain, collagen type I alpha 3 chain, collagen type II alpha 1 chain, collagen type IV alpha 1 chain, collagen type IV alpha 2 chain, collagen type IV alpha 3 chain, collagen type IV alpha 4 chain, collagen type IV alpha 5 chain, collagen type V alpha 1 chain, collagen type V alpha 2 chain, collagen type VI alpha 1 chain, collagen type VI alpha 2 chain, collagen type VI alpha 3 chain, collagen type VIII alpha 1 chain, collagen type XVI alpha 1 chain, and collagen type XXI alpha 1 chain;
wherein the one or more fragments of glycoproteins comprise fibulin 2, periostin, vitronectin, dermatopontin, laminin subunit alpha 3, laminin subunit alpha 5, laminin subunit beta 2, laminin subunit gamma 1, and nidogen 1;
wherein the one or more fragments of proteoglycans comprise biglycan and heparan sulfate proteoglycan core protein;
wherein the one or more fragments of elastins comprise elastin isoform;
wherein the one or more fragments of matrisome secreted factors comprise hornerin;
wherein the one or more fragments of ECM regulators comprise alpha 1 antitrypsin, cathepsin G, desmoplakin, junction plakoglobin, serum albumin precursor, and metalloproteinase inhibitor 3;
wherein the one or more fragments of structure proteins comprise keratin 1, keratin 2, keratin 5, keratin 9, keratin 10, and keratin 14; and
wherein the one or more fragments of growth factors comprise basic fibroblast growth factor, brain-derived neurotrophic factor, epidermal growth factor receptor, endocrine gland-derived vascular endothelial growth factor, growth differentiation factor 15, hepatocyte growth factor, insulin-like growth factor binding protein 1, insulin-like growth factor binding protein 6, osteoprotegerin, platelet-derived growth factor AA, and vascular endothelial growth factor.

21. A method of promoting homeostasis, repair, or regeneration in a target tissue, the method comprising:
topically administering a composition to an epithelium of the target tissue, the composition comprising:
a deconstructed matrisome including one or more enzymatically fragmented peptides derived from at least one biological tissue, and
one or more pharmaceutically acceptable or cosmetically acceptable excipients,
wherein the deconstructed matrisome is in an amount from about 0.1% by weight to about 15% by weight of the composition; and
wherein the one or more enzymatically fragmented peptides are configured to retain cell signaling ability, thereby promoting one or more of tissue homeostasis, tissue repair, and tissue regeneration in the target tissue.

22. The method of claim 21, wherein the tissue exhibits one or more of acute injury, wound, scarring, acne, eczema, and psoriasis.

23. The method of claim 21, wherein topically administering the composition results in an increase in expression of keratins.

24. The method of claim 21, wherein the keratins comprise one or more of keratin 1, keratin 2, keratin 9, and keratin 10.

25. The method of claim 21, wherein topically administering the composition results in an increase in cell regeneration.

26. The method of claim 21, wherein the tissue is a skin tissue, and wherein topically administering the composition results in a decrease in skin redness.

27. The method of claim 21, wherein the tissue comprises a wound, and wherein topically administering the composition results in a decrease in a diameter of the wound.

28. The method of claim 21, wherein the tissue comprises an injury, and wherein topically administering the composition results in a reduction in the formation and appearance of scars.

29. The method of claim 21, wherein the tissue comprises scarring, and wherein topically administering the composition results in an increase in scar healing.

30. A method of increasing keratin gene expression in a target tissue, the method comprising:
topically administering a composition to an epithelium of the target tissue, the composition comprising:
a deconstructed matrisome including one or more enzymatically fragmented peptides derived from at least one biological tissue, and
one or more pharmaceutically acceptable or cosmetically acceptable excipients,
wherein the deconstructed matrisome is in an amount of about 0.1% by weight to about 15% by weight of the composition; and
wherein the one or more enzymatically fragmented peptides are configured to retain cell signaling ability, thereby reducing or preventing one or more of laxity, wrinkling, and sagging in the target tissue.

31. A method of reducing dermal redness in a target tissue, the method comprising:
topically administering a composition to an epithelium of the target tissue, the composition comprising:
a deconstructed matrisome including one or more enzymatically fragmented peptides derived from at least one biological tissue, and
one or more pharmaceutically acceptable or cosmetically acceptable excipients,
wherein the deconstructed matrisome is in an amount of about 0.1% by weight to about 15% by weight of the composition; and
wherein the one or more enzymatically fragmented peptides are configured to retain cell signaling ability, thereby promoting healing or recovery in the target tissue from one or more of scar formation, a wound, and a burn.

32. A method of lowering the pH of a surface of a target tissue, the method comprising:
topically administering a composition to an epithelium of the target tissue, the composition comprising:
a deconstructed matrisome including one or more enzymatically fragmented peptides derived from at least one biological tissue, and
one or more pharmaceutically acceptable or cosmetically acceptable excipients,
wherein the deconstructed matrisome is in an amount of about 0.1% by weight to about 15% by weight of the composition; and
wherein the one or more enzymatically fragmented peptides are configured to retain cell signaling ability, thereby lowering the pH of the surface and reducing presence or growth of a pathogen on the surface of the target tissue.

33. A method of improving a characteristic of a target skin tissue, the method comprising:
topically administering a composition to an epithelium of the target skin tissue, the composition comprising:
a deconstructed matrisome including one or more enzymatically fragmented peptides derived from at least one biological tissue, and
one or more pharmaceutically acceptable or cosmetically acceptable excipients,
wherein the deconstructed matrisome is in an amount of about 0.1% by weight to about 15% by weight of the composition;
wherein the one or more enzymatically fragmented peptides are configured to retain cell signaling ability, thereby improving the characteristic of the target skin tissue; and
wherein the characteristic of the target skin tissue is selected from the group consisting of firmness, elasticity, fine lines, wrinkles, skin texture, skin tone, and appearance.

34. A method of increasing cell regeneration in a target tissue, the method comprising:
topically administering a composition to an epithelium of the tissue, the composition comprising:
a deconstructed matrisome including one or more enzymatically fragmented peptides derived from at least one biological tissue, and
one or more pharmaceutically acceptable or cosmetically acceptable excipients,
wherein the deconstructed matrisome is in an amount of about 0.1% by weight to about 15% by weight of the composition; and
wherein the one or more enzymatically fragmented peptides are configured to retain cell signaling ability, thereby restoring an epithelial barrier of the target tissue.
